SEQUENCE LISTING (12 sequences)

SEQ ID NO: 1 - skipped

SEQ ID NO: 2 - protein, 11 aa, Homo sapiens
PPPLQRAPPPM

SEQ ID NO: 3 - protein, 18 aa, Homo sapiens
APPPMRSKNPGPSQGKSM

SEQ ID NO: 4 - protein, 9 aa, Homo sapiens
  MOD_RES (7)..(7): Phosphorylated-Ser
SKNPGPSQG

SEQ ID NO: 5 - protein, 12 aa, Homo sapiens
  MOD_RES (7)..(7): Phosphorylated-Ser
SKNPGPSQGKSM

SEQ ID NO: 6 - protein, 10 aa, Homo sapiens
  MOD_RES (6)..(6): Symmetrically methylated-Arg
APPPMRSKNP

SEQ ID NO: 7 - protein, 10 aa, Homo sapiens
  MOD_RES (6)..(6): Asymmetrically methylated-Arg
APPPMRSKNP

SEQ ID NO: 8 - protein, 12 aa, Homo sapiens
  MOD_RES (10)..(10): Acetylated-Lys
SKNPGPSQGKSM

SEQ ID NO: 9 - protein, 11 aa, Homo sapiens
  MOD_RES (6)..(6): Asymmetrically methylated-Arg
PPPLQRAPPPM

SEQ ID NO: 10 - protein, 11 aa, Homo sapiens
  MOD_RES (6)..(6): Symmetrically methylated-Arg
PPPLQRAPPPM

SEQ ID NO: 11 - protein, 12 aa, Homo sapiens
  MOD_RES (7)..(7): Phosphorylated-Ser
  MOD_RES (10)..(10): Acetylated-Lys
SKNPGPSQGKSM

SEQ ID NO: 12 - protein, 32 aa, Homo sapiens
QPVAGGMGPPPLQRAPPPMRSKNPGPSQGKSM

(19) United States
(12) Patent Application Publication
(10) Pub. No.: US 2016/0222131 A1
(43) Pub. Date: Aug. 4, 2016

(54) ATRX AS A COMPANION DIAGNOSTIC FOR CDK4 INHIBITORS

(71) Applicant: Memorial Sloan-Kettering Cancer Center, New York, NY (US)

(72) Inventors: Andrew Koff, Westbury, NY (US); Marta Kovatcheva, New York, NY (US); Samuel Singer, New York, NY (US); John Petrini, Brooklyn, NY (US); Mary Elizabeth Klein, New York, NY (US)

(21) Appl. No.: 15/135,401

(22) Filed: Apr. 21, 2016

Related U.S. Application Data
(63) Continuation of application No. PCT/US2014/061621, filed on Oct. 21, 2014.
(60) Provisional application No. 61/893,739, filed on Oct. 21, 2013.

(51) Int. Cl. C07K 16/40; A61K 31/519; G01N 33/50; A61K 31/453; G01N 33/573; C12N 15/113; A61K 31/506

(52) U.S. Cl. CPC C07K 16/40; C12N 15/1137; A61K 31/519; A61K 31/506; A61K 31/453; G01N 33/573; G01N 33/5023; C07K 2317/34; C07K 2317/76; C07K 2317/24; C07K 2317/55; C07K 2317/54; C07K 2317/622; C07K 2317/56; C07K 2317/21; C12N 2310/14; C12N 2310/122; C12N 2310/531; C12N 2310/111; C12N 2320/30; G01N 2333/914

(57) ABSTRACT

The present invention relates to the use of one or more biomarkers to evaluate the likelihood that a CDK4 inhibitor would produce an anti-cancer effect in a subject. It is based, at least in part, on the discovery that cancer cells expressing ATRX prior to treatment are more likely to undergo cellular senescence in response to treatment with a CDK4 inhibitor. It is also based, at least in part, on the discovery that ATRX is phosphorylated in cancer cells that are not as responsive to CDK4 inhibitor therapy. Accordingly, in non-limiting embodiments, the present invention provides for methods, compositions, and kits for a companion diagnostic for CDK4 inhibitors, and in particular, to the use of ATRX expression as a biomarker for determining the likelihood that a cancer can be successfully and/or unsuccessfully treated by CDK4 inhibition.

### PRIORITY CLAIM

This application claims priority to U.S. Provisional Application No. 61/893,739, filed Oct. 21, 2013, the contents of which is hereby incorporated by reference in its entirety herein.

### GRANT INFORMATION

This invention was made with government support under Grant No. P50CA140146 awarded by the National Institutes of Health. The government has certain rights in the invention.

### 1. INTRODUCTION

This present invention relates to ATRX as a biomarker for evaluating the likelihood that a CDK4 inhibitor alone or in addition to another agent would produce an anti-cancer effect in a subject. As such, evaluating ATRX phenotypes may be used as part of a patient treatment protocol.

### 2. BACKGROUND OF THE INVENTION

CDK4 inhibition therapies are currently in numerous U.S. clinical trials for several cancers including liposarcoma, ER-positive HER2-negative breast cancer, lung cancer, multiple myeloma and glioblastoma. Although patients typically are pre-screened for genetic lesions that would render their disease responsive to the drug (i.e., Rb protein expression, CDK4 amplification or loss of CDKN2A depending on the trial), responsiveness to therapy is nonetheless uncertain. For example, in the Phase II trial for liposarcoma carried out at Memorial Sloan-Kettering Cancer Center, approximately 66% of patients achieved progression-free survival that met the trial criteria, while 3% of patients achieved RECIST (Response Evaluation Criteria In Solid Tumors) response. The remaining 31% of patients had progressive disease while being treated with the drug, indicating that predicting the efficacy of these drugs on patient outcome is problematic.

Alpha-thalassemia/mental retardation syndrome X-linked (ATRX) is encoded by the atrx gene. ATRX is a SWI/SNF helicase/ATPase that can regulate gene expression via chromatin remodeling and is associated with pericentric and telomeric heterochromatin (McDowell et al. PNAS 1999; Eustermann et al. NSMB 2011). Its primary clinical indication is mutations in the mental retardation syndrome α-thalassemia/MR, X-linked (ATRX syndrome) (Picketts D J et al. Am J Human Genet 1996). Although ATRX can interact with several proteins that are involved in senescence including PML bodies (Xue et al. PNAS 2003; Luciani et al. J. Cell Science 2006), HP1 proteins (McDowell et al. PNAS 1999; Eustermann et al. NSMB 2011) and macroH2A (Ratnakumar et al. Genes and Dev 2012), ATRX has never been directly associated with senescence. Studies have shown that ATRX negatively regulates macroH2A (a facilitator of senescence-associated heterochromatic foci formation) incorporation into chromatin (Ratnakumar et al. Genes and Dev 2012).

### 3. SUMMARY OF THE INVENTION

The present invention relates to methods and compositions which provide a companion diagnostic for CDK4 inhibitors, and in particular, to the use of ATRX as a biomarker for the likelihood that a cancer can be successfully treated by CDK4 inhibition. It is based, at least in part, on the discovery that protein extracts of cancer cells that undergo cellular senescence in response to treatment with a CDK4 inhibitor contain an ATRX protein that binds to a certain ATRX antibody with a much greater affinity than ATRX protein in cancer cells that are not as responsive or unresponsive to CDK4 inhibitor therapy. It is also based, at least in part, on the discovery that ATRX is phosphorylated in cancer cells that are not as responsive or unresponsive to CDK4 inhibitor therapy.

### 4. BRIEF DESCRIPTION OF THE FIGURES

FIG. 1. ATRX was detected by Western blot using the Bethyl antibody in responder cell lines but was not detectable in non-responder cell lines. Immunoblots were used to measure the amount of ATRX and DAXX. γ-Tubulin was used as a loading control. This experiment was repeated at least three times for each cell line.

FIG. 2A-B. shRNA generates effective knockdown in the responder cell line LS8817. Immunoblot and immunofluorescence were used to identify suitable shRNA targeting vectors that would reduce the accumulation of ATRX. This experiment was repeated at least three times.

FIG. 3. Reducing ATRX levels in the responder cell line LS8817 was sufficient to maintain MDM2 and bypass senescence in response to PD0322991 treatment. The effect of reducing ATRX on PD0332991 induced changes in BrdU incorporation, senescence-associated β-gal, and MDM2 accumulation in the responder cell line LS8817 were analyzed.

FIG. 4A-B. Reducing ATRX levels attenuated the capacity of MDM2 knockdown to induce senescence in the responder cell line LS8817. MDM2 was subsequently reduced in responder (A) and non-responder cells (B) in which ATRX was initially reduced and the effect on senescence was determined. This experiment was repeated at least three times.

FIG. 5. ATRX status was not associated with alternative lengthening of telomeres (ALT) in responder and non-responder cell lines as measured by telomeric restriction fragment length assay.

FIG. 6. ATRX status was not associated with ALT in responder and non-responder cell lines as measured by telomeric fluorescence in situ hybridization (FISH).

FIG. 7. ATRX was detectable by Western blot using the Bethyl antibody in glioma and breast cancer cell lines.

FIG. 8A-B. (A) Reducing ATRX levels in the responder cell line LS8817 was sufficient to maintain MDM2. (B) Reducing ATRX levels in the responder cell line L8817 prevented the rapid decrease in MDM2 protein observed in response to CDK4 inhibition.

FIG. 9. Using the D5 antibody, ATRX was detected in all WD/DDLS cell lines compared to ATRX detected by the Bethyl antibody.

FIG. 10. A modified form of ATRX is present in non-responder cell lines that is not detected by the Bethyl antibody.

FIG. 11. The specificity of the Bethyl antibody was tested against 8 peptides that correspond to certain amino acids within the ATRX protein sequence.

FIG. 12. ATRX phosphorylated at amino acid S2487 was detected in non-responder cells.

FIG. 13A-B. (A) The number of ATRX foci was increased in each senescent cell induced by MDM2 knockdown. (B) The mean number of nuclear ATRX foci in each cell did not increase in non-responder cells in response to PD0332991 treatment, but did in responder cells in response to PD0332991 treatment.

FIG. 14. The number of ATRX foci in responder cells undergoing senescence was significantly more than in responder cells that did not undergo treatment with PD0332991. Six independent experiments are shown. The average number of foci per cell in each experiment is indicated. The percentage of cells with greater than 18 foci is also shown.

FIG. 15. The average number of ATRX foci per cell in non-responder cells did not increase significantly upon treatment with PD0332991 when compared with non-responders that were not treated with PD0332991. Six independent experiments are shown. The average number of foci per cell in each experiment is indicated. The percentage of cells with greater than 10 foci is also shown.

FIG. 16A-C. The increase in the number of ATRX foci in response to PD0332991 treatment in responder cells was observed as early as 2 days after treatment with PD0332991. The number of ATRX foci in responder cells was determined 2 days, 4 days and 7 days after treatment with PD0332991 (A-B). The results of the two experiments of A and B were combined in (C).

FIG. 17 depicts a non-limiting embodiment of an assay for determining whether a non-responder cell undergoes senescence in response to treatment with PD0332991 in conjunction with a second drug according to the present invention.

FIG. 18. Treatment of cells with PD0332991 followed by shMDM2 resulted in a significant increase in the number of ATRX foci within non-responders cells compared to cells that were not treated with PD0332991 or were treated with PD0332991 alone.

### 5. DETAILED DESCRIPTION OF THE INVENTION

For clarity and not by way of limitation the detailed description of the invention is divided into the following subsections:
(i) ATRX as a biomarker;
(ii) anti-ATRX antibodies;
(iii) CDK4 inhibitors;
(iv) cancer targets;
(v) methods of use; and
   (a) therapeutic methods using the '045 antibody;
   (b) therapeutic methods using the antibodies disclosed in section 5.2;
   (c) therapeutic methods using the ATRX P-S2487 biomarker;
   (d) assay.
(vi) kits.

### 5.1 ATRX as a Biomarker

Alpha-thalassemia/mental retardation syndrome X-linked is denoted ATRX herein.

In certain non-limiting embodiments, an ATRX biomarker may be a protein.

In a specific, non-limiting embodiment, an ATRX protein may be a human ATRX protein having the amino acid sequence as set forth in NCBI database accession no. NP_000480.

ATRX proteins for non-human species are known or can be determined according to methods known in the art, for example, where the sequence is the allele represented in the majority of the population.

In a specific, non-limiting embodiment, an ATRX protein may be a mouse ATRX protein having the amino acid sequence as set forth in NCBI database accession no. NP_033556.

In a specific, non-limiting embodiment, an ATRX protein may be a rat ATRX protein having the amino acid sequence as set forth in NCBI database accession no. XP_003754859.

In non-limiting embodiments, an ATRX biomarker can be an ATRX protein that is detectably present, using an antibody directed toward ATRX (“ATRX-specific antibody”).

In non-limiting embodiments, an ATRX biomarker can be an ATRX protein that is detectably present, using an ATRX-specific antibody sold by Bethyl, Catalog No. A301-045A (“the '045 Ab”), a fragment thereof, or an antibody that competitively inhibits binding of the '045 Ab to ATRX, in cells that are responsive to treatment with a CDK4 inhibitor (“Responder Cells”).

A Responder Cell is a cancer cell which, when treated with an effective amount of a CDK4 inhibitor, increases expression of one or more markers of the senescent phenotype, including, but not limited to SA-β-gal, senescence-associated heterochromatin foci and elaboration of the senescence-associated secretory program and/or increases the number of ATRX foci in the nucleus (see below) and/or exhibits a decrease in MDM2 protein, relative to the level without treatment with the CDK4 inhibitor. In non-limiting embodiments, the mean level of nuclear ATRX foci increase may be at least 30 percent and/or the level of decrease of MDM2 may be at least about 10 percent.

A Non-responder Cell is a cancer cell which is not a Responder Cell. In certain non-limiting embodiments, a Non-responder Cell, when treated with an amount of a CDK4 inhibitor effective in inducing senescence in Responder Cells, does not increase expression of at least one marker, or at least two markers, or at least three markers, of the senescent phenotype selected from the group consisting of sA-β-gal, senescence-associated heterochromatin foci and elaboration of the senescence-associated secretory program and/or does not increase the number of ATRX foci in the nucleus (see below) and/or exhibits stable or increased levels of MDM2 protein, relative to the level without treatment with the CDK4 inhibitor.

In non-limiting embodiments, an ATRX biomarker can be an ATRX protein that is detectably present, using an ATRX-specific antibody disclosed in Section 5.2, or a fragment thereof, in Non-responder Cells, and is not detectably present in Responder Cells. In non-limiting embodiments, an ATRX biomarker may be a human ATRX protein having the amino acid sequence as set forth in NCBI database accession no. NP_000480 that is post-translationally modified. For example, and not by way of limitation, an ATRX biomarker that is detectable in Non-responder Cells can be an ATRX protein that is phosphorylated at amino acid S2487 (“P-S2487”). In non-limiting embodiments, an ATRX biomarker that is detectable in Non-responder Cells can be an ATRX protein that is symmetrically methylated at amino acid R2480 (“R2480”).

The term “detectable” as used herein with reference to an ATRX biomarker means that an index or signal produced by a detection agent, for example an antibody, is apparent under standard experimental conditions. For example, but not by way of limitation, an ATRX biomarker is “detectable” if, in a Western blot of proteins prepared from cancer cells, an index or signal produced by a detection agent, for example an ATRX-specific antibody, is present under conditions in which a positive control protein, such as γ-tubulin, is detectable. In non-limiting embodiments, an ATRX biomarker is “detectable” if, in a Western blot of proteins prepared from cancer cells, the signal resulting from binding of an ATRX-specific antibody is at least 0.1 times the signal resulting from binding of an antibody directed toward γ-tubulin, bound and visualized under essentially the same conditions. In non-limiting examples, the antibody directed toward γ-tubulin may be C20 antibody from Santa Cruz Biotechnology

The term “not detectable” or “not detected” or “undetected” as used herein with reference to ATRX means that an index or signal produced by a detection agent, for example an antibody, is substantially absent under conditions in which a positive control protein, such as γ-tubulin, is detectable. According to this definition it is possible that a low signal may be present even when ATRX would be considered, according to the invention, to be “undetectable.” For example, and not by way of limitation, ATRX is “undetectable” if, in a Western blot of proteins prepared from cancer cells, the signal resulting from binding of an ATRX-specific antibody is less than 0.02 times and/or less than 0.1 times the signal resulting from binding of an antibody directed toward a housekeeping protein, e.g., γ-tubulin, bound and visualized under essentially the same conditions. In non-limiting embodiments, the ATRX-specific antibody is the antibody sold by Bethyl, Catalog No. A301-045A (“the '045 Ab”), a fragment thereof, or an antibody that competitively inhibits binding of the '045 Ab to ATRX and is not the D5 antibody sold by Santa Cruz Biotechnology, Catalog No. sc-55584. In non-limiting embodiments, the ATRX-specific antibody is an anti-ATRX antibody disclosed in Section 5.2, or a fragment thereof. In non-limiting embodiments, the antibody directed toward γ-tubulin may be C20 antibody from Santa Cruz Biotechnology.

In non-limiting embodiments of the invention, ATRX is detectable if it is detectable in an immunoblot prepared and performed as set forth in the Example section (section 6.1) below.

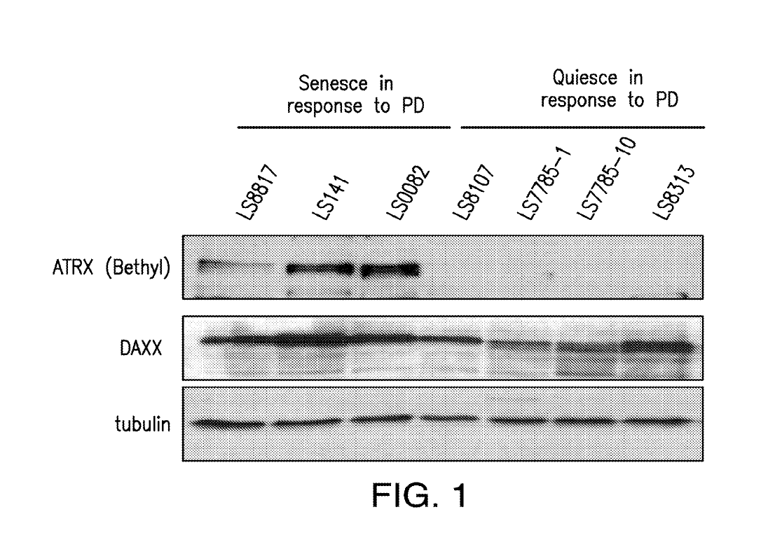

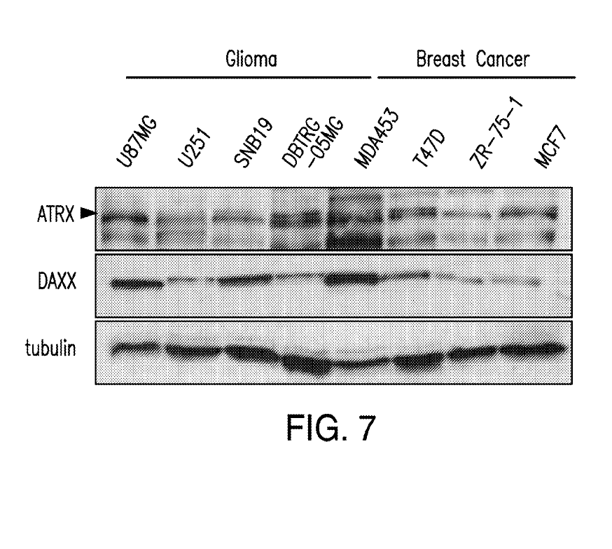

In non-limiting embodiments of the invention, ATRX is detectably present with the use of the '045 Ab in the following cell lines: LS8817, LS141, LS0082, U87MG (ATCC HTB-14), U251 (Cat#09063001, Sigma-Aldrich), SNB19 (Cat# ACC 325, DSMZ), DBTRG-05MG (ATCC CRL-2020), MDA-MB-453 (ATCC HTB-131), T-47D (ATCC HTB-133), ZR-75-1 (ATCC CRL-1500), and MCF7 (ATCC HTB-22) (see FIGS. 1 and 7).

In non-limiting embodiments of the invention, ATRX is not detectably present with the use of the '045 Ab in the following cell lines: LS8107, LS7785-1, LS7785-10, LS8313, G-292 (ATCC CRL-1423), and U-2 OS (ATCC HTB-96) (see FIG. 1).

A reference control level of ATRX using the '045 Ab may, for example, be established using cancer cells that are not responsive to a CDK4 inhibitor.

A reference control level of ATRX using the ATRX-antibodies disclosed in section 5.2, e.g., Ab3 and Ab4, may, for example, be established using cancer cells that are responsive to a CDK4 inhibitor.

Methods for detecting and/or determining the level of ATRX include, but are not limited to, mass spectrometry techniques, 1-D or 2-D gel-based analysis systems, chromatography, protein microarray, immunofluorescence, enzyme linked immunosorbent assays (ELISAs), radioimmunoassay (RIA), enzyme immunoassays (EIA), Western Blotting and other immunoglobulin-mediated assays, and other techniques known in the art.

In non-limiting embodiments, methods of ATRX detection may utilize the ATRX-specific antibody sold by Bethyl, Catalog No. A301-045A (“the '045 Ab”), a fragment thereof, or an antibody that competitively inhibits binding of the '045 Ab to ATRX.

Alternatively or additionally, methods of ATRX detection may utilize one or more ATRX antibodies disclosed in section 5.2, or a fragment thereof, or an antibody that competitively inhibits binding of the antibodies disclosed in section 5.2 with ATRX. In certain embodiments, ATRX can be detected using an ATRX-specific antibody that binds to an ATRX protein phosphorylated at amino acid S2487 (“P-S2487”) and is not the D5 antibody sold by Santa Cruz Biotechnology, Catalog No. se-55584.

Methods of detection used herein preferably do not utilize the D5 antibody sold by Santa Cruz Biotechnology, Catalog No. sc-55584 because that antibody detects ATRX in both responder and non-responder cells.

In certain, non-limiting embodiments, one method that can be used for detecting an ATRX biomarker is Western blotting. Cells can be harvested by trypsinization and homogenized and/or sonicated in lysis buffer. Lysates can be clarified by centrifugation and subjected to SDS-PAGE followed by transfer to a membrane, such as a polyvinylidene difluoride (PVDF) membrane. Antibodies (unlabeled), specific to a biomarker, i.e., ATRX, can then brought into contact with the membrane and assayed by a secondary immunological reagent, such as labeled anti-immunoglobulin. Non-limiting examples of labels include, but are not limited to, ¹²⁵I, horseradish peroxidase and alkaline phosphatase. In certain embodiments, immunodetection can be performed with antibody to a biomarker using the enhanced chemiluminescence system (e.g., from PerkinElmer Life Sciences, Boston, Mass.). The membrane can then be stripped and re-blotted with a control antibody, e.g., anti-tubulin.

In certain, non-limiting embodiments, immunohistochemistry can be used for detecting an ATRX biomarker. For example, an antibody can be brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The labeling can be scored visually using microscopy and the results can be quantitated.

### 5.2 Anti-ATRX Antibodies

The present invention provides antibodies specific for ATRX. For example, and not by way of limitation, the present invention provides antibodies that bind post-translationally modified forms of ATRX.

The term “antibody” or “antibodies,” as used herein, refers to all types of immunoglobulins, including IgG, IgM, IgA, IgD, and IgE, including Fab or antigen-recognition fragments thereof. The disclosed antibodies may be monoclonal or polyclonal and may be of any species of origin, including, but not limited to, mouse, rat, rabbit, horse or human, or chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984). The antibodies may be recombinant monoclonal antibodies produced according to the methods disclosed in U.S. Pat. No. 4,474,893 or U.S. Pat. No. 4,816,567.

5.2.1 Exemplary Anti-ATRX Antibodies

In non-limiting embodiments of the present invention, the disclosed antibodies bind to an epitope within the C-terminus domain of the ATRX protein, e.g., an ATRX-specific antibody. The term “epitope,” as used herein, refers to a protein determinant capable of specific binding to an antibody. Epitopes usually consist of chemically active surface groupings of molecules such as amino acids or sugar side chains and usually have specific three-dimensional structural characteristics, as well as specific charge characteristics.

In a non-limiting embodiment, an anti-ATRX antibody of the present invention binds to an ATRX epitope within a region of ATRX comprising the amino acids 2242-2492 of the amino acid sequence set forth in NCBI database accession no. NP_000480 (SEQ ID NO:1).

In a non-limiting embodiment, an anti-ATRX antibody of the present invention binds to an epitope within a fragment of ATRX comprising the amino acid sequence PPPLQRAPPPM (SEQ ID NO:2; “Ab1”), or fragment thereof. In certain non-limiting embodiments, the amino acid sequence of SEQ ID NO:2 corresponds to amino acids 2469-2479 of ATRX.

In a non-limiting embodiment, an anti-ATRX antibody of the present invention binds to an epitope within a fragment of ATRX comprising the amino acid sequence APPPMRSKNPGPSQGKSM (SEQ ID NO:3; “Ab2”), or fragment thereof. In certain non-limiting embodiments, the amino acid sequence of SEQ ID NO:3 corresponds to amino acids 2475-2492 of ATRX.

In a non-limiting embodiment, an anti-ATRX antibody of the present invention can bind to an ATRX protein that is phosphorylated at amino acid S2487 and is not the D5 antibody sold by Santa Cruz Biotechnology, Catalog No. sc-55584. For example, and not by way of limitation, an anti-ATRX antibody of the present invention binds to an epitope within a fragment of ATRX comprising the amino acid sequence SKNPGPSQG, or fragment thereof, where S is phosphorylated (SEQ ID NO:4; “Ab3”). In certain non-limiting embodiments, the amino acid sequence of SEQ ID NO:4 corresponds to amino acids 2481-2489 of ATRX. In certain embodiments, an anti-ATRX antibody of the present invention binds to an epitope within a fragment of ATRX comprising the amino acid sequence SKNPGPSQGKSM, or fragment thereof, where S is phosphorylated (SEQ ID NO:5; “Ab4”). In certain non-limiting embodiments, the amino acid sequence of SEQ ID NO:5 corresponds to amino acids 2481-2492 of ATRX.

The present invention provides an anti-ATRX antibody that binds to an ATRX protein, or a fragment thereof, that is phosphorylated at amino acid S2487 and does not bind to a ATRX protein that is not phosphorylated at amino acid S2487, where the anti-ATRX antibody is not the D5 antibody by Santa Cruz Biotechnology, Catalog No. sc-55584.

The present invention provides an anti-ATRX antibody that binds to an ATRX protein, or a fragment thereof, that is phosphorylated at amino acid S2487 and is not the D5 antibody by Santa Cruz Biotechnology, Catalog No. sc-55584.

In certain non-limiting embodiments, anti-ATRX antibody of the present invention can bind to an ATRX protein that is dimethylated at amino acid R2480, and is not the D5 antibody sold by Santa Cruz Biotechnology, Catalog No. sc-55584. For example, and not by way of limitation, an anti-ATRX antibody of the present invention binds to an epitope within a fragment of ATRX comprising the amino acid sequence APPPMRSKNP, or fragment thereof, where R is symmetrically dimethylated (SEQ ID NO:6; “Ab5”), In certain embodiments, an anti-ATRX antibody of the present invention binds to an epitope within a fragment of ATRX corresponding to amino acids 2475-2484 of ATRX comprising the amino acid sequence APPPMRSKNP, or fragment thereof, where R is asymmetrically dimethylated (SEQ ID NO:7; “Ab6”). In non-limiting embodiments, the amino acid sequences of SEQ ID NOs:6 and 7 correspond to amino acids 2475-2484 of ATRX.

In certain non-limiting embodiments, anti-ATRX antibody of the present invention can bind to an ATRX protein that is acetylated at amino acid K2490. In certain embodiments, an anti-ATRX antibody of the present invention binds to an epitope within a fragment of ATRX comprising the amino acid sequence SKNPGPSQGKSM, or fragment thereof, where K is acetylated (SEQ ID NO:8; “Ab7”). In certain non-limiting embodiments, the amino acid sequence of SEQ. ID NO:8 corresponds to amino acids 2481-2492 of ATRX.

In certain non-limiting embodiments, anti-ATRX antibody of the present invention can bind to an ATRX protein that is methylated at amino acid R2474. In certain embodiments, an anti-ATRX antibody of the present invention binds to an epitope within a fragment of ATRX comprising the amino acid sequence PPPLQRAPPPM, or fragment thereof, where R is asymmetrically dimethylated (SEQ ID NO:9; “Ab8”). In certain embodiments, an anti-ATRX antibody of the present invention binds to an epitope within a fragment of ATRX comprising the amino acid sequence PPPLQRAPPPM, or fragment thereof, where R is symmetrically dimethylated (SEQ ID NO:10; “Ab9”). In non-limiting embodiments, the amino acid sequences of SEQ ID NOs:9 and 10 correspond to amino acids 2469-2479 of ATRX.

In certain non-limiting embodiments, anti-ATRX antibody of the present invention can bind to an ATRX protein that is acetylated at amino acid 2490 and phosphorylated at amino acid 2487. In certain embodiments, an anti-ATRX antibody of the present invention binds to an epitope within a fragment of ATRX comprising the amino acid sequence SKNPGPSQGKSM, where S is phosphorylated and K is acetylated (SEQ ID NO:11; “Ab10”), or fragment thereof. In certain non-limiting embodiments, the amino acid sequence of SEQ ID NO:11 corresponds to amino acids 2481-2492 of ATRX.

The present invention further provides antibodies that were raised to one or more peptides having the amino acid sequences of SEQ ID NO:1, 2, 3, 4, 5, 6, 7, 8, 9, 10 or 11. For example, and not by way of limitation, the present invention provides antibodies that were generated by immunization of an animal, disclosed above, with a peptide having the amino acid sequence of SEQ ID NO:1, 2, 3, 4, 5, 6, 7, 8, 9, 10 or 11. In non-limiting embodiments, an ATRX-specific antibody of the present invention was generated by immunization of an animal with a peptide having the amino acid sequence of SEQ ID NO:4, or a fragment thereof. In non-limiting embodiments, an ATRX-specific antibody of the present invention was generated by immunization of an animal with a peptide having the amino acid sequence of SEQ ID NO:5, or a fragment thereof. In non-limiting embodiments, an ATRX-specific antibody of the present invention was generated by immunization of an animal with a peptide having the amino acid sequence of SEQ ID NO:6, or a fragment thereof. In non-limiting embodiments, an ATRX-specific antibody of the present invention was generated by immunization of an animal with a peptide having the amino acid sequence of SEQ ID NO:4, followed by the immunization of the same animal with a peptide having the amino acid sequence of SEQ ID NO:5.

In certain embodiments, an anti-ATRX antibody of the present invention can have a dissociation constant (K_{d}) from about 0.001 nM to about 1 μM or from about 10⁻⁸M to about 10⁻¹³M. For example, and not by way of limitation, the anti-ATRX antibody can have a K_{d}≦1 μM, <100 nM, <10 nM, ≦1 nM, ≦0.1 nM, ≦0.01 nM or ≦0.001 nM. In certain embodiments, the anti-ATRX antibody can have a K_{d} of about 10⁻⁸M or less, from about 10⁻⁹M or less, about 10⁻¹⁰M or less, about 10⁻¹¹ M or less, about 10⁻¹²M or about 10⁻¹³ M or less to ATRX, e.g., human ATRX.

In non-liming embodiments, polyclonal antibodies of the present invention may be produced according to standard techniques by immunizing a suitable animal (e.g., mouse, rabbit, goat, etc.) with an antigen, i.e., peptide, encompassing a fragment of the amino acid sequence of ATRX described herein (e.g., a peptide having an amino acid sequence of SEQ ID NOs:1-11), collecting immune serum from the animal and separating the polyclonal antibodies from the immune serum, in accordance with known procedures.

In non-liming embodiments, monoclonal antibodies of the present invention may be produced in a hybridoma cell line according to the well-known technique of Kohler and Milstein, Nature 265: 495-97 (1975); Kohler and Milstein, Eur. J. Immunol. 6: 511 (1976); see also, Current Protocols In Molecular Biology, Ausubel et al. Eds. (1989). Monoclonal antibodies produced by such a method are highly specific, and improve the selectivity and specificity of the methods disclosed herein. For example, ant not by way of limitation, a solution containing the appropriate antigen, i.e., peptide (e.g., a peptide having an amino acid sequence of SEQ ID NOs:1-11), may be injected into a mouse or other species and, after a sufficient time (in keeping with conventional techniques), the animal is sacrificed and spleen cells obtained. The spleen cells can then immortalized by fusing them with myeloma cells, typically in the presence of polyethylene glycol, to produce hybridoma cells. The hybridoma cells can then be grown in a suitable selection media, and the supernatant screened for monoclonal antibodies having the desired specificity, as described below. The secreted antibody may be recovered from tissue culture supernatant by conventional methods such as centrifugation, precipitation, ion exchange or affinity chromatography, or the like.

In non-liming embodiments, an antibody or antigen-binding portion thereof of the present invention can tested for its antigen binding activity, e.g., by known methods such as ELISA, Western blot, etc. Alternatively and/or additionally, competition assays may be used to identify an anti-ATRX antibody of the present invention that cross-competes with the Bethyl antibody for binding to ATRX (e.g., human ATRX).

In a non-limiting embodiment of a competition assay, immobilized ATRX is incubated in a solution comprising a first labeled antibody that binds to ATRX (e.g., Bethyl antibody) and a second unlabeled antibody, e.g., Ab1 or Ab2, that is being tested for its ability to compete with the first antibody for binding to ATRX. The second antibody may be present in a hybridoma supernatant. As a control, immobilized ATRX is incubated in a solution comprising the first labeled antibody but not the second unlabeled antibody. After incubation under conditions permissive for binding of the first antibody to ATRX, excess unbound antibody is removed, and the amount of label associated with immobilized ATRX is measured. If the amount of label associated with immobilized ATRX is substantially reduced in the test sample relative to the control sample, then that indicates that the second antibody is competing with the first antibody for binding to ATRX. See Harlow and Lane (1988) Antibodies: A Laboratory Manual ch.14 (Cold Spring Harbor Laboratory, Cold Spring Harbor, N.Y.).

5.2.2 Antibody Fragments

The present invention provides antigen-binding portions, or antibody fragments, of anti-ATRX antibodies. Antigen-binding portions or antibody fragments include, but are not limited to, Fab, Fab′, Fab′-SH, F(ab′)₂, Fv and scFv fragments (see Hudson et al. Nat. Med. 9:129-134 (2003); Pluckthün, in The Pharmacology of Monoclonal Antibodies, vol. 113, Rosenhurg and Moore eds., (Springer-Verlag, New York), pp. 269-315 (1994); PCT Application No. WO 93/16185; and U.S. Pat. Nos. 5,571,894 and 5,587,458.

Antibody fragments can be made by various techniques, including, but not limited to, proteolytic digestion of an intact antibody as well as production by recombinant host cells (e.g., E. coli or phage), as described herein.

5.2.3 Chimeric and Humanized Antibodies

The present invention further provides anti-ATRX chimeric antibodies. Certain chimeric antibodies are described, e.g., in U.S. Pat. No. 4,816,567 and in Morrison et al., Proc. Natl. Acad. Sci. USA, 81:6851-6855 (1984). In non-liming embodiments, a chimeric antibody comprises a non-human variable region (e.g., a variable region derived from a mouse, rat, hamster, rabbit or non-human primate, such as a monkey) and a human constant region. In non-liming embodiments, a chimeric antibody can be a “class switched” antibody in which the class or subclass has been changed from that of the parent antibody. Chimeric antibodies include antigen-binding fragments thereof.

In non-liming embodiments, an anti-ATRX chimeric antibody is a humanized antibody. A non-human antibody can be humanized to reduce immunogenicity to humans, while retaining the specificity and affinity of the parental non-human antibody. A humanized antibody can include one or more variable domains in which heavy variable regions (HVRs), complementarity determining regions (CDRs), or portions thereof, are derived from a non-human antibody, and framework regions (FRs), or portions thereof, are derived from human antibody sequences. In certain non-limiting embodiments, a humanized antibody can also include at least a portion of a human constant region. In non-liming embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are disclosed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005); Padlan, Mol. Immunol. 28:489-498 (1991); Dall'Acqua et al., Methods 36:43-60 (2005); and Osbourn et al., Methods 36:61-68 (2005) and Klimka et al., Br. J. Cancer, 83:252-260 (2000).

Human framework regions that may be used for humanization include, but are not limited to, framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem, 271:22611-22618 (1996)).

5.2.4 Human Antibodies

In certain embodiments, an anti-ATRX antibody provided herein is a human antibody. Human antibodies can be produced using various techniques known in the art. Human antibodies are disclosed, generally, in van Dijk and van de Winkel, Curr. Opin. Pharmacol. 5: 368-74 (2001) and Lonberg, Curr. Opin. Immunol. 20:450-459 (2008).

Human antibodies can be prepared by administering an immunogen, e.g., a peptide, to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. See, for example, Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals can be further modified, e.g., by combining with a different human constant region.

In certain embodiments, human antibodies can also be made by hybridoma-based methods. Human myeloma and mouse-human heteromyeloma cell lines for the production of human monoclonal antibodies have been described in Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

In certain embodiments, human antibodies can also be generated by isolating Fv clone variable domain sequences selected from human-derived phage display libraries. Such variable domain sequences may then be combined with a desired human constant domain.

5.2.5 Labeled Antibodies

The present invention further provides labeled anti-ATRX antibodies. Non-limiting examples of labels include labels or moieties that are detected directly (such as fluorescent, chromophoric, electron-dense, chemiluminescent and radioactive labels), as well as moieties, such as enzymes or ligands, that are detected indirectly, e.g., through an enzymatic reaction or molecular interaction. For example, and not by way of limitation, labels can include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (see U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals and the like.

### 5.3 CDK4 Inhibitors

Non-limiting examples of CDK4 inhibitors include compounds that inhibit the kinase activity of CDK4. Additional non-limiting examples of CDK4 inhibitors include ATP-competitive inhibitors of CDK4. In particular non-limiting embodiments, the CDK4 inhibitor is derived from pyridopyrimidine or indolocarbazole compounds. Further non-limiting examples of CDK4 inhibitors include Palbociclib Isethionate, LEE011, LY2835219, PD0332991, and Flavopiridol Hydrochloride. Additional CDK4 inhibitors are disclosed in U.S. Pat. Nos. 6,630,464 and 6,818,663, and U.S. Patent Application No. U.S. 2012/244,110.

Further non-limiting examples of CDK4 inhibitors include antisense oligonucleotides, shRNA molecules, and siRNA molecules that specifically inhibit the expression or activity of CDK4. One non-limiting example of a CDK4 inhibitor comprises an antisense, shRNA, or siRNA nucleic acid sequence homologous to at least a portion of a CDK4 nucleic acid sequence, wherein the homology of the portion relative to the CDK4 sequence is at least about 75 or at least about 80 or at least about 85 or at least about 90 or at least about 95 or at least about 98 percent, where percent homology can be determined by, for example, BLAST or FASTA software. In certain non-limiting embodiments, the complementary portion may constitute at least 10 nucleotides or at least 15 nucleotides or at least 20 nucleotides or at least 25 nucleotides or at least 30 nucleotides and the antisense nucleic acid, shRNA or siRNA molecules may be up to 15 or up to 20 or up to 25 or up to 30 or up to 35 or up to 40 or up to 45 or up to 50 or up to 75 or up to 100 nucleotides in length. Antisense, shRNA, or siRNA molecules may comprise DNA or atypical or non-naturally occurring residues, for example, but not limited to, phosphorothioate residues.

### 5.4 Cancer Targets

Non-limiting examples of cancers that may be subject to the present invention include soft tissue sarcomas, liposarcoma, glioma (or glioblastoma), basal cell carcinoma, melanoma, lung cancer and breast cancer.

### 5.5 Methods of Use

In certain non-limiting embodiments, the present invention provides for a method of determining whether an anti-cancer effect is likely to be produced in a cancer of a subject by a CDK4 inhibitor, comprising, determining the expression level of an ATRX biomarker in a cancer. In certain non-limiting embodiments, the method can include determining the expression level of an ATRX biomarker in a cancer cell sample. In non-limiting embodiments, the method can include determining the expression level of an ATRX protein biomarker.

A subject may be human or a non-human subject. Non-limiting examples of non-human subjects include non-human primates, dogs, cats, mice, rats, guinea pigs, rabbits, pigs, fowl, horses, cows, goats, sheep, cetaceans, etc.

An anti-cancer effect means one or more of a reduction in aggregate cancer cell mass, a reduction in cancer cell growth rate, a reduction in cancer cell proliferation, a reduction in tumor mass, a reduction in tumor volume, a reduction in tumor cell proliferation, a reduction in tumor growth rate, a reduction in tumor metastasis and/or an increase in the proportion of senescent cancer cells.

In certain non-limiting embodiments, a sample includes, but is not limited to, a clinical sample, cells in culture, cell supernatants, cell lysates, serum, blood plasma, biological fluid (e.g., lymphatic fluid), and tissue samples. The source of the sample may be solid tissue (e.g., from a fresh, frozen, and/or preserved organ, tissue sample, biopsy, or aspirate) or cells from the individual, including circulating tumor cells. In certain non-limiting embodiments, the sample is obtained from a tumor.

In non-limiting embodiments, the ATRX biomarker is an ATRX protein that is specifically detected using the '045 Ab, a fragment thereof, or an antibody that competitively inhibits the binding of the '045 Ab to ATRX. In certain embodiments, the ATRX biomarker is an ATRX protein that is specifically detected using an antibody disclosed in section 5.2 above, e.g., Ab3 or Ab4, or a fragment thereof.

ATRX biomarkers that can be detected using the '045 Ab or the antibodies disclosed in section 5.2 are described in the sections above. CDK4 inhibitors are described above. Cancers suitable for treatment are described above. Methods for determining the expression level of an ATRX biomarker are set forth in section 5.1 above.

In certain non-limiting embodiments, the method can include the treatment of the subject with a therapeutically effective amount of a CDK4 inhibitor and/or a therapeutically effective amount of another modality, for example, an alternative chemotherapeutic agent, biologic anticancer agent or radiation therapy. A “therapeutically effective amount” is an amount that is able to achieve one or more of an anticancer effect, prolongation of survival and/or prolongation of period until relapse.

5.5.1 Therapeutic Methods Using the Bethyl Antibody (the '045 Ab)

The present invention provides for a method of determining whether an anti-cancer effect is likely to be produced in a cancer by a CDK4 inhibitor by determining the level of an ATRX biomarker that is detectable by the ATRX-specific antibody sold by Bethyl, Catalog No. A301-045A (“the '045 Ab”), a fragment thereof, or an antibody that competitively inhibits binding of the '045 Ab to ATRX.

In certain non-limiting embodiments, the method of determining whether an anti-cancer effect is likely to be produced in a cancer by a CDK4 inhibitor, comprises, determining the expression level of an ATRX biomarker in a cancer using the '045 Ab, or fragment thereof, where the detection of the ATRX biomarker indicates that it is more likely that the CDK4 inhibitor would have an anti-cancer effect on the cancer. In other non-limiting embodiments, where an ATRX biomarker is not detected using the '045 Ab, or fragment thereof, it is less likely that the CDK4 inhibitor would have an anti-cancer effect on the tumor (for example, relative to cancers where ATRX is detectable). In certain non-limiting embodiments, the ATRX-specific antibody can be an antibody that competitively inhibits the binding of the '045 Ab to ATRX, as disclosed above.

In certain, non-limiting embodiments, the detection of the ATRX biomarker may be appreciated by comparing the level of the ATRX biomarker in the cancer prior to CDK4 inhibitor treatment to a reference standard as described above. For example, and not by way of limitation, detection of the ATRX biomarker can be appreciated by comparing the level of the ATRX biomarker to the level of ATRX biomarker in one or more Non-responder cell lines, e.g., LS8107.

In certain non-limiting embodiments, the present invention provides for a method for determining whether an anti-cancer effect is likely to be produced in a cancer by a CDK4 inhibitor, comprising, obtaining one or more samples of the cancer before treatment with a CDK4 inhibitor, and determining, in the one or more sample, the expression level of an ATRX biomarker using the '045 Ab, or fragment thereof, where if the ATRX biomarker is detected, it is more likely that a CDK4 inhibitor would have an anti-cancer effect on the cancer. As stated supra, the detection of an ATRX biomarker may be appreciated by comparing the expression level of an ATRX biomarker in the cancer prior to CDK4 inhibitor treatment to a reference control standard.

In certain non-limiting embodiments, the present invention provides for a method for producing an anti-cancer effect by a CDK4 inhibitor in a subject, comprising, obtaining a sample of the cancer before treatment of the subject with a CDK4 inhibitor, and determining, in one or more cancer cell from the sample, the expression level of an ATRX biomarker using the '045 Ab, or fragment thereof, where if the expression of the ATRX biomarker is detected prior to treatment with the CDK4 inhibitor, then initiating treatment of the subject with a therapeutically effective amount of the CDK4 inhibitor.

In certain non-limiting embodiments, the present invention provides for a method for producing an anti-cancer effect by a CDK4 inhibitor, comprising, obtaining a sample of the cancer after treatment with a CDK4 inhibitor, and determining, in one or more cancer cell from the sample, the expression level of an ATRX biomarker using the '045 Ab, or fragment thereof, where if expression of the ATRX biomarker is detected following treatment with a CDK4 inhibitor, then continuing or resuming treatment of the subject with a therapeutically effective amount of a CDK4 inhibitor. Optionally, a sample may be collected before and after treatment and the ATRX levels can be monitored and/or compared.

In certain non-limiting embodiments, the CDK4 inhibitor used to treat a subject after the detection of an ATRX biomarker in a pre-treatment sample may be the same or different from the CDK4 inhibitor administered after the detection of the ATRX biomarker in a post-treatment sample. In certain non-limiting embodiments, the CDK4 inhibitor used to treat a subject after the detection of an ATRX biomarker in a pre-treatment sample may be from the same or different chemical class than the CDK4 inhibitor administered after the detection of the ATRX biomarker in a post-treatment sample. In certain non-limiting embodiments, the CDK4 inhibitor used to treat a subject after the detection of an ATRX biomarker in a pre-treatment sample may function by a similar or different mechanism than the CDK4 inhibitor administered after the detection of the ATRX biomarker in a post-treatment sample.

In certain non-limiting embodiments, the present invention provides for a method for treating a subject having a cancer, comprising obtaining a sample of the cancer before treatment of the subject with a CDK4 inhibitor, and determining, in one or more cancer cell from the sample the expression level of an ATRX biomarker using the '045 Ab, or fragment thereof, where if the expression of the ATRX biomarker is detected prior to treatment with the CDK4 inhibitor, then initiating treatment of the subject with a therapeutically effective amount of the CDK4 inhibitor.

In certain non-limiting embodiments, the present invention provides for a method for treating a subject having a cancer, comprising obtaining a sample of the cancer after treatment of the subject with a CDK4 inhibitor, and determining, in one or more cancer cell from the sample the expression level of an ATRX biomarker using the '045 Ab, or fragment thereof, where if expression of the ATRX biomarker is detected following treatment with a CDK4 inhibitor, then continuing or resuming treatment of the subject with a therapeutically effective amount of a CDK4 inhibitor. Optionally, a sample may be collected before and after treatment and the ATRX levels can be monitored and/or compared.

Any of the foregoing methods may comprise a step of collecting one or more cancer cell samples from the subject, where a cell or cells from the subject may be used to determine the ATRX biomarker level in the cancer cell sample.

In certain non-limiting embodiments, where an ATRX biomarker specifically detected by the '045 Ab is not detectable in cancer cells prior to treatment with a CDK4 inhibitor, the subject from whom the cancer cells derive is treated with another modality, for example, an alternative chemotherapeutic agent, biologic anticancer agent or radiation therapy.

5.5.2 Therapeutic Methods Using the ATRX Antibodies of Section 5.2

The present invention provides for a method of determining whether an anti-cancer effect is likely to be produced in a cancer by a CDK4 inhibitor by determining the level of an ATRX biomarker that is detectable by an antibody disclosed in section 5.2, or a fragment thereof.

In certain non-limiting embodiments, the method of determining whether an anti-cancer effect is likely to be produced in a cancer by a CDK4 inhibitor, comprises, determining the expression level of an ATRX biomarker in a cancer using an ATRX-specific antibody disclosed in section 5.2 such as Ab3, Ab4, Ab5 or Ab10, where the detection of the ATRX biomarker indicates that it is less likely that the CDK4 inhibitor would have an anti-cancer effect on the cancer.

In certain non-limiting embodiments, the ATRX antibody for use in the disclosed methods is specific for ATRX phosphorylated at amino acid S2487. In certain non-limiting embodiments, the ATRX, antibody for use in the disclosed methods is specific for ATRX phosphorylated at amino acid S2487 and is not the D5 antibody sold by Santa Cruz Biotechnology, Catalog No. sc-55584. For example, the antibody specific for ATRX phosphorylated at amino acid 52487 can be Ab3 or Ab4.

In certain non-limiting embodiments, the ATRX antibody can be specific for ATRX symmetrically methylated at amino acid R2480. In certain non-limiting embodiments, the ATRX antibody for use in the disclosed methods is specific for ATRX symmetrically methylated at amino acid R2480 and is not the D5 antibody sold by Santa Cruz Biotechnology, Catalog No. sc-55584. In certain non-limiting embodiments, the ATRX antibody for use in the disclosed methods is specific for ATRX symmetrically methylated at amino acid R2480 and does not bind to ATRX that is not methylated or asymmetrically methylated at amino acid R2480. For example, the antibody specific for ATRX symmetrically methylated at amino acid R2480 can be Ab5.

In other non-limiting embodiments, where the ATRX marker is not detected using an ATRX antibody disclosed in section 5.2 such as, but not limited to, Ab3, Ab4, Ab5 or Ab10, it is more likely that the CDK4 inhibitor would have an anti-cancer effect on the tumor. In certain, non-limiting embodiments, the detection of the ATRX biomarker may be appreciated by comparing the level of the ATRX biomarker in the cancer prior to CDK4 inhibitor treatment to a reference standard as described above.

In certain non-limiting embodiments, the present invention provides for a method for determining whether an anti-cancer effect is likely to be produced in a cancer by a CDK4 inhibitor, comprising, obtaining one or more samples of the cancer before treatment with a CDK4 inhibitor, and determining, in the one or more samples, the expression level of an ATRX biomarker using an ATRX antibody disclosed in section 5.2 such as, but not limited to, Ab3, Ab4, Ab5 or Ab10, where if the expression of the ATRX biomarker is detected, it is less likely that a CDK4 inhibitor would have an anti-cancer effect on the cancer.

In certain non-limiting embodiments, the present invention provides for a method for producing an anti-cancer effect by a CDK4 inhibitor in a subject, comprising, obtaining a sample of the cancer before treatment of the subject with a CDK4 inhibitor, and deter mining, in one or more cancer cell from the sample, the expression level of an ATRX biomarker using an ATRX antibody Ab3, Ab4 or Ab5, where if the expression of the ATRX biomarker is detected prior to treatment with the CDK4 inhibitor, then initiating treatment of the subject with another modality, for example, an alternative chemotherapeutic agent, biologic anticancer agent or radiation therapy. For example, and not way of limitation, if the ATRX biomarker in the cancer sample is detectable by Ab3 or Ab4, which is specific to ATRX phosphorylated at S2487, treatment with another modality can include treatment with a protein kinase inhibitor that prevents phosphorylation of ATRX.

In certain non-limiting embodiments, treatment with another modality can include treatment with one or more inhibitors for ATM, ATR and/or DNA-PK. Non-limiting examples of ATM inhibitors include KU55933 and KU59403. Non-limiting examples of ATR inhibitors include VE-822 and schisandrin B. Non-limiting examples of DNA-PK inhibitors include wortmannin, NK314, OK-1035, SU11752, LY294002 and LY294002 derivatives such as IC86621, IC87102, IC87361, NU7026 and NU7441. Additional non-limiting examples of ATM, ATR and DNA-PK inhibitors are disclosed in Khalil et al. BioDiscovery (2012) and Davidson et al. Frontiers in Pharmacology (2013).

In certain non-limiting embodiments, the present invention provides for a method for producing an anti-cancer effect by a CDK4 inhibitor in a subject, comprising, obtaining a sample of the cancer before treatment of the subject with a CDK4 inhibitor, and determining, in one or more cancer cell from the sample, the expression level of an ATRX biomarker using an ATRX antibody such as Ab3, Ab4, Ab5 or Ab10, where if the expression of the ATRX biomarker is not detected prior to treatment with the CDK4 inhibitor, then initiating treatment of the subject with a therapeutically effective amount of the CDK4 inhibitor.

In certain non-limiting embodiments, the present invention provides for a method for producing an anti-cancer effect by a CDK4 inhibitor, comprising, obtaining a sample of the cancer after treatment with a CDK4 inhibitor, and determining, in one or more cancer cell from the sample, the expression level of an ATRX biomarker using an ATRX antibody such as Ab3, Ab4, Ab5 or Ab10, where if expression of the ATRX biomarker is detected following treatment with a CDK4 inhibitor, then initiating treatment of the subject with another modality, for example, an alternative chemotherapeutic agent, biologic anticancer agent or radiation therapy. Optionally, a sample may be collected before and after treatment and the ATRX levels using an ATRX antibody such as Ab3, Ab4, Ab5 or Ab10 can be monitored and/or compared.

In certain non-limiting embodiments, the present invention provides for a method for treating a subject having a cancer, comprising obtaining a sample of the cancer before treatment of the subject with a CDK4 inhibitor, and determining, in one or more cancer cell from the sample the expression level of an ATRX biomarker using an ATRX antibody such as Ab3, Ab4, Ab5 or Ab10, where if the expression of the ATRX biomarker is detected prior to treatment with the CDK4 inhibitor, then initiating treatment of the subject with a therapeutically effective amount of the CDK4 inhibitor.

In certain non-limiting embodiments, the present invention provides for a method for treating a subject having a cancer, comprising obtaining a sample of the cancer after treatment of the subject with a CDK4 inhibitor, and determining, in one or more cancer cell from the sample the expression level of an ATRX biomarker using an ATRX antibody such as Ab3, Ab4, Ab5 or Ab10, where if expression of the ATRX biomarker is detected following treatment with a CDK4 inhibitor, then stopping treatment of the subject with a therapeutically effective amount of a CDK4 inhibitor. Alternatively and/or additionally, the method can include treatment of the subject with a therapeutically effective amount of a CDK4 inhibitor in combination with treatment with another modality, for example, an alternative chemotherapeutic agent, biologic anticancer agent or radiation therapy, as disclosed above. Optionally, a sample may be collected before and after treatment and the ATRX levels can be monitored and/or compared.

“In combination with” or “in conjunction with,” as used interchangeably herein, means that the CDK4 inhibitor and the other modality are administered to a subject as part of a treatment regimen or plan. In certain embodiments, being used in combination does not require that the CDK4 inhibitor and the other modality are physically combined prior to administration or that they be administered over the same time frame. For example, and not by way of limitation, the CDK4 inhibitor and the other modality can be administered concurrently to the subject being treated, or can be administered at the same time or sequentially in any order or at different points in time.

Any of the foregoing methods may comprise a step of collecting one or more cancer cell sample from the subject, where a cell or cells from the subject may be used to determine the ATRX biomarker level in the cancer cell sample.

5.5.3 Therapeutic Methods Using the ATRX P-S2487 Biomarker

The present invention provides for a method of determining whether an anti-cancer effect is likely to be produced in a cancer by a CDK4 inhibitor by determining the level of an ATRX biomarker, where the ATRX biomarker is an ATRX protein phosphorylated at S2487 (“P-S2487”). Methods of detecting ATRX protein phosphorylated at S2487 used herein preferably do not utilize the D5 antibody sold by Santa Cruz Biotechnology, Catalog No. sc-55584 because that antibody detects ATRX in both responder and non-responder cells.

In certain non-limiting embodiments, the method of determining whether an anti-cancer effect is likely to be produced in a cancer by a CDK4 inhibitor, comprising, determining the expression level of an ATRX biomarker in a cancer, where the ATRX biomarker is an ATRX protein phosphorylated at S2487, and where the detection of the ATRX biomarker indicates that it is less likely that the CDK4 inhibitor would have an anti-cancer effect on the cancer.

In certain non-limiting embodiments, the present invention provides for a method for determining whether an anti-cancer effect is likely to be produced in a cancer by a CDK4 inhibitor, comprising, obtaining one or more samples of the cancer before treatment with a CDK4 inhibitor, and determining, in the one or more samples, the expression level of an ATRX biomarker, where the ATRX biomarker is an ATRX protein phosphorylated at S2487, and where if the expression of the ATRX biomarker is detected, it is less likely that a CDK4 inhibitor would have an anti-cancer effect on the cancer.

In certain non-limiting embodiments, the present invention provides for a method for producing an anti-cancer effect by a CDK4 inhibitor in a subject, comprising, obtaining a sample of the cancer before treatment of the subject with a CDK4 inhibitor, and determining, in one or more cancer cell from the sample, the expression level of an ATRX biomarker, where the ATRX biomarker is an ATRX protein phosphorylated at S2487, and where if the expression of the ATRX biomarker is detected prior to treatment with the CDK4 inhibitor, then initiating treatment of the subject with another modality, for example, an alternative chemotherapeutic agent, biologic anticancer agent or radiation therapy, as disclosed above.

In certain non-limiting embodiments, the present invention provides for a method for producing an anti-cancer effect by a CDK4 inhibitor in a subject, comprising, obtaining a sample of the cancer before treatment of the subject with a CDK4 inhibitor, and determining, in one or more cancer cell from the sample, the expression level of an ATRX biomarker, where the ATRX biomarker is an ATRX protein phosphorylated at S2487, and where if the expression of the ATRX biomarker is not detected prior to treatment with the CDK4 inhibitor, then initiating treatment of the subject with a therapeutically effective amount of the CDK4 inhibitor.

In certain non-limiting embodiments, the present invention provides for a method for producing an anti-cancer effect by a CDK4 inhibitor, comprising, obtaining a sample of the cancer after treatment with a CDK4 inhibitor, and determining, in one or more cancer cell from the sample, the expression level of an ATRX biomarker, where the ATRX biomarker is an ATRX protein phosphorylated at S2487, and where if expression of the ATRX biomarker is detected following treatment with a CDK4 inhibitor, then initiating treatment of the subject with another modality, for example, an alternative chemotherapeutic agent, biologic anticancer agent or radiation therapy. Optionally, a sample may be collected before and after treatment and the ATRX levels can be monitored and/or compared.

In certain non-limiting embodiments, the present invention provides for a method for treating a subject having a cancer, comprising obtaining a sample of the cancer before treatment of the subject with a CDK4 inhibitor, and determining, in one or more cancer cell from the sample the expression level of an ATRX biomarker, where the ATRX biomarker is an ATRX protein phosphorylated at S2487, and where if the expression of the ATRX biomarker is detected prior to treatment with the CDK4 inhibitor, then initiating treatment of the subject with a therapeutically effective amount of the CDK4 inhibitor.

In certain non-limiting embodiments, the present invention provides for a method for treating a subject having a cancer, comprising obtaining a sample of the cancer after treatment of the subject with a CDK4 inhibitor, and determining, in one or more cancer cells from the sample the expression level of an ATRX biomarker, where the ATRX biomarker is an ATRX protein phosphorylated at S2487, and where if expression of the ATRX biomarker is detected following treatment with a CDK4 inhibitor, then stopping treatment of the subject with a therapeutically effective amount of a CDK4 inhibitor. Alternatively and/or additionally, the method can include treatment of the subject with a therapeutically effective amount of a CDK4 inhibitor in combination with treatment with another modality, for example, an alternative chemotherapeutic agent, biologic anticancer agent or radiation therapy, as disclosed above. Optionally, a sample may be collected before and after treatment and the ATRX levels can be monitored and/or compared.

Any of the foregoing methods may comprise a step of collecting one or more cancer cell sample from the subject, where a cell or cells from the subject may be used to determine the ATRX biomarker level in the cancer cell sample.

5.5.4 Assay

The present invention further provides an assay for identifying other modalities, e.g., chemotherapeutic drugs, that can be used in conjunction with a CDK4 inhibitor for treatment of a subject with cancer by analyzing the number of ATRX foci observed per cell following treatment with the modality. In non-limiting embodiments, the assay can be used to identify cells that may be responsive to CDK4 inhibition alone. As discussed in the Examples below, an increase in the number of ATRX foci per cell is observed in cells that are undergoing senescence and in senescent cells, and is observed in cells that are responsive to CDK4 inhibition.

The disclosed assay provides a high-throughput screening method for identifying modalities that can be used in conjunction with a CDK4 inhibitor to increase the sensitivity of cancer cells to CDK4 inhibition and result in senescence. For example, and not by way of limitation, the disclosed assay can allow screening of large libraries of compounds. One non-limiting example of a library is an FDA approved library of compounds that can be used by humans.

In certain non-limiting embodiments, the assay includes treating one or more cells that do not undergo senescence in response to CDK4 inhibition with a CDK4 inhibitor. Non-limiting examples of CDK4 inhibitors are disclosed above. For example, and not by way of limitation, the CDK4 inhibitor for use in the disclosed assay can be PD03322991. The amount of CDK4 inhibitor applied to the cells depends on the type of CDK4 inhibitor used. In non-limiting embodiments, cells can be treated with a CDK4 inhibitor at a concentration of about 100 nM to about 10 μM. For example, and not by way of limitation, the cells can be treated with a CDK4 inhibitor at a concentration from about 100 nM to about 5 μM, from about 100 nM to about 2 μM, from about 100 nM to about 1 μM, from about 500 nM to about 10 μM, from about 500 nM to about 5 μM, from about 500 nM to about 2 μM, from about 500 nM to about 1 μM, from about 750 nM to about 10 μM, from about 750 nM to about 5 μM, from about 750 nM to about 2 μM, or from about 750 nm to about 1 μM.

The cells for use in the disclosed assay can be any cell type that does not undergo senescence in response to CDK4 inhibition but can exit the cell cycle and are Rb-positive. For example, and not by way of limitation, the cells for use in the disclosed assay can include LS8107, LS7785-1, LS7785-10, LS8313, H358 and H3122. In a non-limiting embodiment, the cells can be LS8107 cells. In non-limiting embodiments, the cells can be cancer cells from a patient, or a population of cells cultured from cancer cells from a patient. For example, and not by way of limitation, the cells can be derived from a cancer within a patient that is not responsive to CDK4 inhibition.

The assay provided herein may be performed in multiwell formats, in microtiter plates, in multispot formats or in arrays. In certain non-limiting embodiments, the cells for use in the present invention can be cultured and grown in 96-well microtiter plates.

After treatment with a CDK4 inhibitor, one or more treated cells can subsequently be treated with a second modality, e.g., a candidate compound. In non-limiting embodiments, the cells can be treated with a second modality one day, two days, three days, four days, five days, six days, seven days, eight days or more after treatment with the CDK4 inhibitor. In a specific non-limiting embodiment, the one or more cells can be treated with the second modality two days after treatment with the CDK4 inhibitor.

Candidate compounds to be screened in the currently disclosed assay can be any pharmacologic agents already known in the art or can be compounds previously unknown to have any pharmacological activity. Non-limiting examples of drug candidates can include known drugs such as those described in well-known literature references such as the Merck Index, the Physicians Desk Reference and The Pharmacological Basis of Therapeutics. For example, and not by way of limitation, the drug candidates can include medicaments; vitamins; mineral supplements; substances used for the treatment and/or prevention of cancer; or prodrugs, which become biologically active or more active after they have been placed in a physiological environment. Additional non-limiting examples of drug candidates include small molecules, antibiotics, antivirals, antifungals, enediynes, heavy metal complexes, hormone antagonists, non-specific (non-antibody) proteins, sugar oligomers, aptamers, oligonucleotides (e.g., antisense oligonucleotides that bind to a target nucleic acid sequence (e.g., mRNA sequence)), siRNA, shRNA, peptides, proteins, radionuclides, and transcription-based pharmaceuticals. In non-limiting embodiments, potential drug candidates can include nucleic acids, peptides, small molecule compounds (e.g., pharmaceutical compounds), and peptidomimetics. Candidate compounds can be naturally occurring compounds or synthetic compounds. For example, and not by way of limitation, the candidate compounds can be isolated from microorganisms, animals or plants, or can be produced recombinantly or synthesized by chemical methods known in the art.

Following treatment with the second modality, e.g., candidate compound, the assay can further include determining the number of ATRX foci per cell, where an increase in the number of ATRX foci per cell in response to treatment with the second modality indicates that the second modality may be useful when administered in combination with a CDK4 inhibitor during the treatment of a subject that has cancer. In non-limiting embodiments, determining the number of ATRX foci can be performed within two days, three days, four days, five days, six days, seven days, eight days or more after treatment with the second modality.

In non-limiting embodiments, an increase in the number of ATRX foci may be appreciated by comparing the number of ATRX foci per cell in the non-responder cells following treatment with the second modality to a reference standard. In certain, non-limiting embodiments, the reference sample can include non-responsive cells that have been treated with the CDK4 inhibitor alone. In certain, non-limiting embodiments, the reference sample can include non-responsive cells that have been treated with the second modality alone. In non-limiting embodiments, an increase in the percentage of cells that have a number of ATRX foci that increase approximately 50% or more per cell on average compared to that of CDK4 inhibitor alone is indicative that the second modality may be useful in combination with a CDK4 inhibitor.

In certain, non-limiting embodiments, immunohistochemistry can be used for detecting ATRX foci. For example, an ATRX-specific antibody can be brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The number of ATRX foci per cell can be scored visually using microscopy and the results can be quantitated. In a specific non-limiting embodiment, the '045 antibody can be used to detect ATRX foci.

In non-limiting embodiments, the assay can include treating one or more cancer cells with a CDK4 inhibitor and, determining, in the one or more CDK4 inhibitor-treated cancer cells, the number of ATRX foci per cell compared to a reference control, where if the number of ATRX foci per cell increases in response to CDK4 inhibition than it is more likely that the CDK4 inhibitor would have an anti-cancer effect on the one or more cancer cells. For example, and not by way of limitation, the one or more cancer cells can be obtained from a patient. In non-limiting embodiments, the reference control can be one or more cancer cells that were not treated with a CDK4 inhibitor, e.g., one or more cancer cells from the patient that was not treated with a CDK4 inhibitor.

### 5.6 Kits

In non-limiting embodiments, the present invention provides for a kit for determining whether an anti-cancer effect is likely to be produced in a cancer by a CDK4 inhibitor, comprising a means for detecting the expression level of a biomarker. ATRX biomarkers and methods for measuring ATRX biomarker levels are described in the sections above.

Types of kits include, but are not limited to, arrays/microarrays, biomarker-specific antibodies and beads, which further contain one or more probes, antibodies or other detection reagents for detecting one or more biomarker of the present invention.

In non-limiting embodiments, the present invention provides for a kit for determining whether the anti-cancer effect is likely to be produced in a cancer by a CDK4 inhibitor, comprising a means for detecting the protein levels of a biomarker.

In non-limiting embodiments, a kit may comprise at least one antibody for immunodetection of the biomarker(s) to be identified. Antibodies, both polyclonal and monoclonal, including molecules comprising an antibody variable region or a subregion thereof, specific for an ATRX biomarker, may be prepared using conventional immunization techniques, as will be generally known to those of skill in the art. In non-limiting embodiments, a kit of the present invention can comprise the ATRX-specific antibody sold by Bethyl, Catalog No. A301-045A (“the '045 Ab”), a fragment thereof, or an antibody that competitively inhibits binding of the '045 Ab to ATRX. Alternatively or additionally, a kit of the present invention can comprise an antibody disclosed in section 5.2. For example, and not by way of limitation, the kit can include Ab1, Ab2, Ab3, Ab4, Ab5, Ab6, Ab7, Ab8, Ab9 and/or Ab10. In non-limiting embodiments, the kit can include Ab3, Ab4, Ab5, Ab10 or combinations thereof.

The immunodetection reagents of the kit may include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5, or ROX), radiolabels (3H, 35S, 32P, 14C, 131I) or enzymes (alkaline phosphatase, horseradish peroxidase). Alternatively, a detectable moiety may be comprised in a secondary antibody or antibody fragment which selectively binds to the first antibody or antibody fragment (where said first antibody or antibody fragment specifically recognizes ATRX).

In a further non-limiting embodiment, the ATRX biomarker-specific antibody may be provided bound to a solid support, such as a column matrix, an array, or a well of a microtiter plate. Alternatively, the support may be provided as a separate element of the kit.

In one specific non-limiting embodiment, a kit may comprise a probe, microarray, or antibody suitable for detecting an ATRX biomarker.

In certain non-limiting embodiments, where the measurement means in the kit employs an array, the set of biomarkers set forth above may constitute at least 10 percent or at least 20 percent or at least 30 percent or at least 40 percent or at least 50 percent or at least 60 percent or at least 70 percent or at least 80 percent of the species of markers represented on the microarray.

In certain non-limiting embodiments, a biomarker detection kit may comprise one or more detection reagents and other components (e.g., a buffer, enzymes such as alkaline phosphatase, antibodies, and the like) necessary to carry out an assay or reaction to determine the expression levels of a biomarker.

A kit may further include instructions for using the kit to determine the expression level of the ATRX biomarker. In non-limiting embodiments, the instructions describes that the detection of an ATRX biomarker using the '045 Ab, set forth herein, is indicative of an increased likelihood of an anti-cancer effect in a cancer by a CDK4 inhibitor. Alternatively or additionally, the instructions can describe that the detection of an ATRX biomarker using an ATRX antibody such as Ab3 and/or 4, is indicative of an decreased likelihood of an anti-cancer effect in a cancer by a CDK4 inhibitor

### 6. EXAMPLE 1

### Senescence in Response to CDK4 Inhibition Requires ATRX

### 6.1 Materials and Methods

Immunoblot.

Cells were harvested by trypsinization and washed twice with PBS. Pellets were then lysed in an equal volume of buffer composed of 50 mM Tris-HCl, pH7.4, 250 mM NaCl, 5 mM EDTA, 0.5% NP40, 2 mM PMSF, and supplemented with protease inhibitors. Pellets were sonicated 2×45 seconds (with at least 1 minute on ice in between each sonication) using a Fisher Scientific Sonic Dismembrator Model 500. Lysates were clarified by spinning for 10 minutes at max speed in a table top microcentrifuge at 4 degrees Celsius. 40 micrograms of protein was resolved on a 6% SDS-PAGE gel. The gel was subsequently transferred onto a PVDF membrane by semi-dry transfer at 34 mA for 150 minutes. Membranes were blocked in 5% milk dissolved in 1×TNT for one hour and then incubated with anti-ATRX antibody at a concentration of 1:2000 (stock was provided at 1 mg/ml), obtained from Bethyl, Cat No. A301-045A, or the anti-ATRX antibody sold by Santa Cruz Biotechnology, Catalog No. sc-55584, (“D5 antibody”) overnight. The following day, membranes were washed 3×15 minutes in 1×TNT, incubated for 1 hour at room temperature with anti-rabbit-HRP secondary antibody (diluted to 1:10,000 in 1×TNT+0.5% tween; stock was provided at 1 mg/ml), washed again 3×15 minutes and signal was developed using standard ECL reagents.

Immunofluorescence.

Cells were washed twice with PBS and fixed with 4% paraformaldehyde at room temperature for 20 minutes. Cells were again washed twice with PBS and treated with 0.1% triton X-100 in PBS for five minutes. Cells were again washed twice with PBS and blocked in 5% BSA in PBS for 30 minutes at room temperature. Cells were incubated with anti-ATRX antibody, obtained from Bethyl, Cat No. A301-045A, diluted at 1:2000 in PBS (stock is provided at 1 mg/ml) overnight at 4 degrees Celsius. The following day, cells were washed twice with PBS and incubated with Alexa Fluor-488 anti-rabbit secondary diluted at 1:500 in PBS (stock was provided at 2 mg/ml) for one hour at room temperature. Cells were washed twice with PBS and incubated with 0.1 ug/ml DAN for 5 minutes at room temperature. Cells were washed three times with PBS and slides were mounted in Vectashield fluorescence mounting media. Slides were imaged using a Zeiss Axioplan 2 Upright Microscope.

### 6.2 Results

Senescence is perceived as a favorable clinical outcome due to its ability to inhibit tumor progression. Rb-positive liposarcoma cell lines have the capacity to undergo either cell cycle arrest or cell cycle arrest with activation of a senescence program in response to CDK4 inhibition. In soft tissue sarcomas such as liposarcoma, as well as other types of tumors (reviewed in Johnson J E and Broccoli D, Curr Opin Oncol 2007), the phenomenon known as alternative lengthening of telomeres (ALT), which is a mechanism by which cells maintain their telomeres independent of telomerase, is quite common. ALT is used by cancer cells as a mechanism of immortalization independent of telomerase activity.

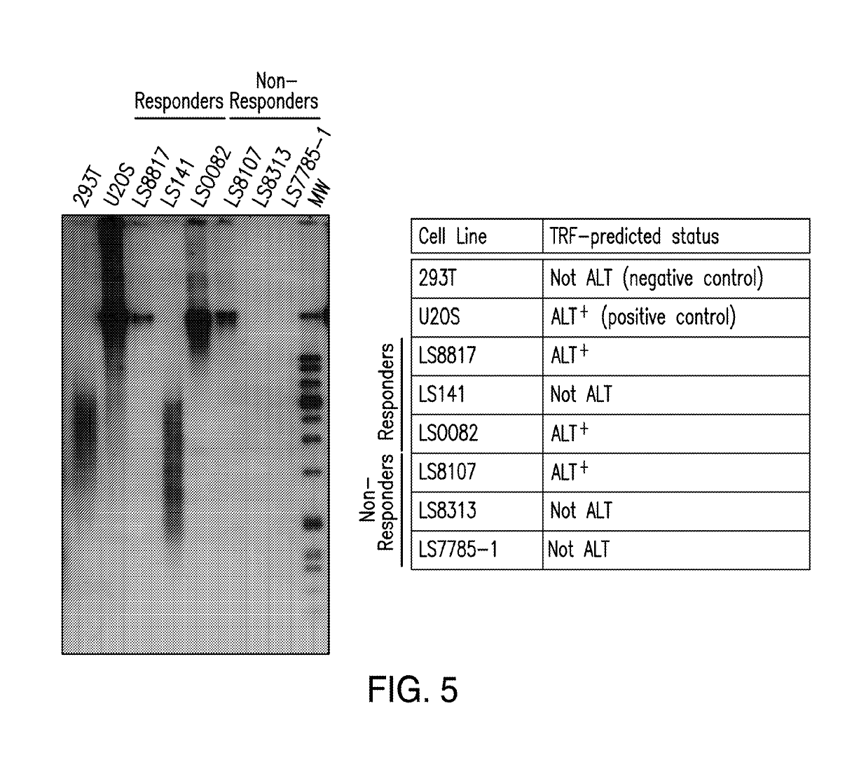

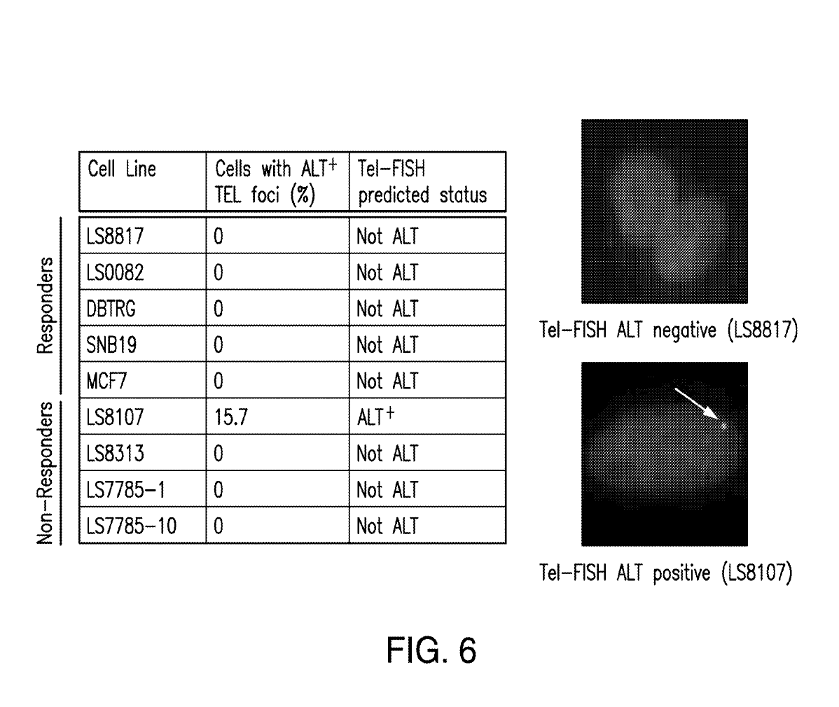

To determine whether ALT plays a role in senescence induced by treatment with the CDK4 inhibitor, PD0332991, ALT was measured in responder and non-responder cancer cell lines. Cell lines that under growth arrest are referred to as non-responder cells and cells that undergo growth arrest that is associated with the induction of senescence are referred to as responder cells. ALT can be detected either via accumulation of high-molecular weight telomeric DNA in the terminal restriction fragment (TRF) southern blotting assay or by the presence of a few large, brightly staining foci as measured by telomeric FISH (Henson et al. Clinical Cancer Res 2005, Slatter et al. American Journal of Pathology 2010). ALT, as measured either by telomere FISH (FIG. 6) or by telomere restriction fragment length (FIG. 5), did not correlate with the definition of a responder in any of the cell types analyzed.

ALT-positive cells are further characterized by a loss or mutation of Alpha-thalassemia/mental retardation syndrome X-linked (ATRX) protein expression (in 90% of ALT-positive cells), a failed capacity to repair double strand breaks and elongated telomeres (Lovejoy et al. PLoS Genetics 2012; Heaphy et al. Science 2011). To determine if ATRX expression is altered in responder cells compared to non-responder cells, ATRX protein was analyzed. ATRX was found to be exclusively detected using the Bethyl antibody by Western blot in the responder liposarcoma cell lines, LS8817, LS141 and LS0082, and in the breast and glioma cell lines, U87MG, U251, SNB19, DBTRG-05MG, MDA453, T-47D, ZR-75-1 and MCF7 (FIGS. 1 and 7). No correlation was observed between loss of ATRX protein and the ALT phenotype, as measured by both TRF and telomeric-FISH in the responder and non-responder cells (FIGS. 5 and 6). Without being bound to a specific theory, these results suggest that ATRX detected by the Bethyl antibody might be involved in the senescence pathway induced by treatment with a CDK4 inhibitor, and may be acting in a way that was unrelated to ALT.

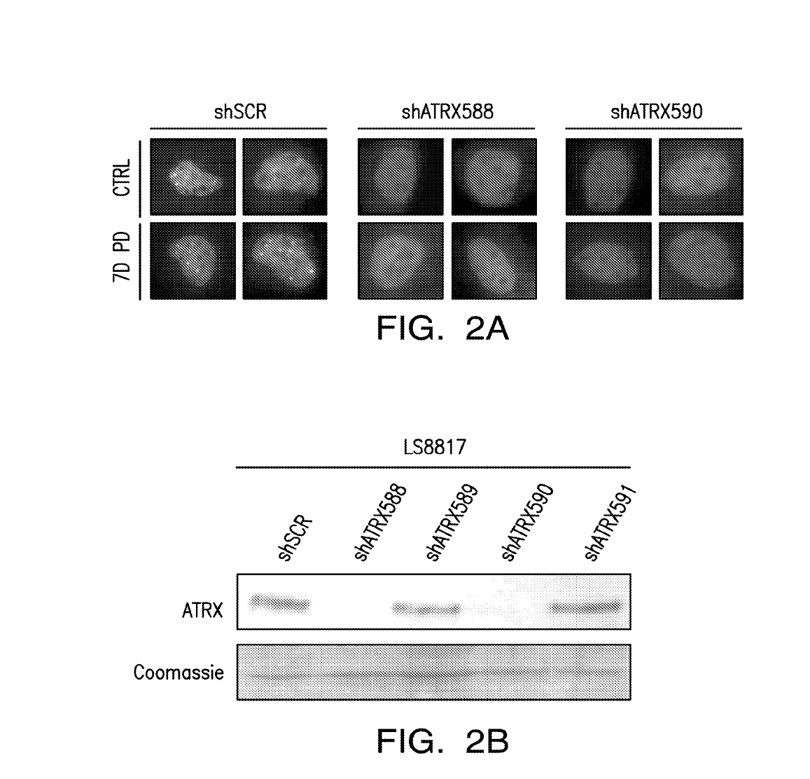

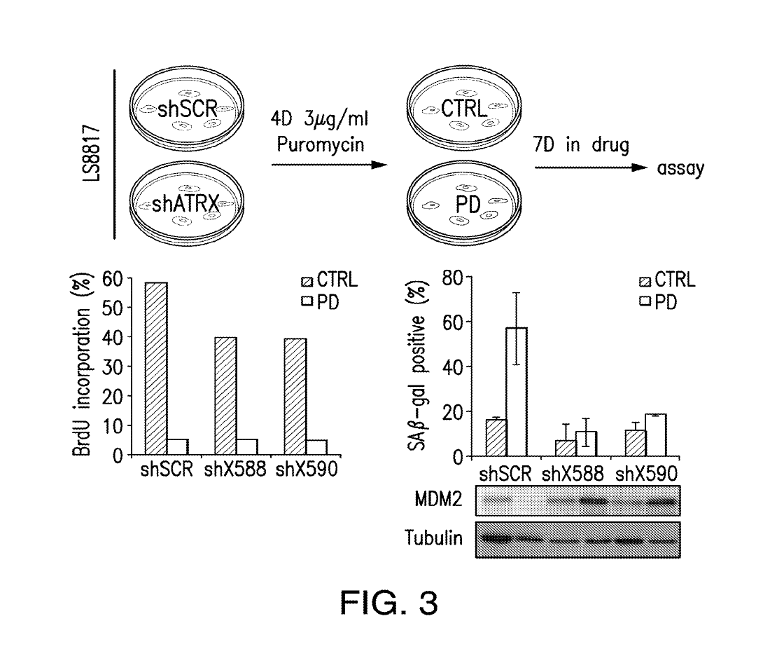

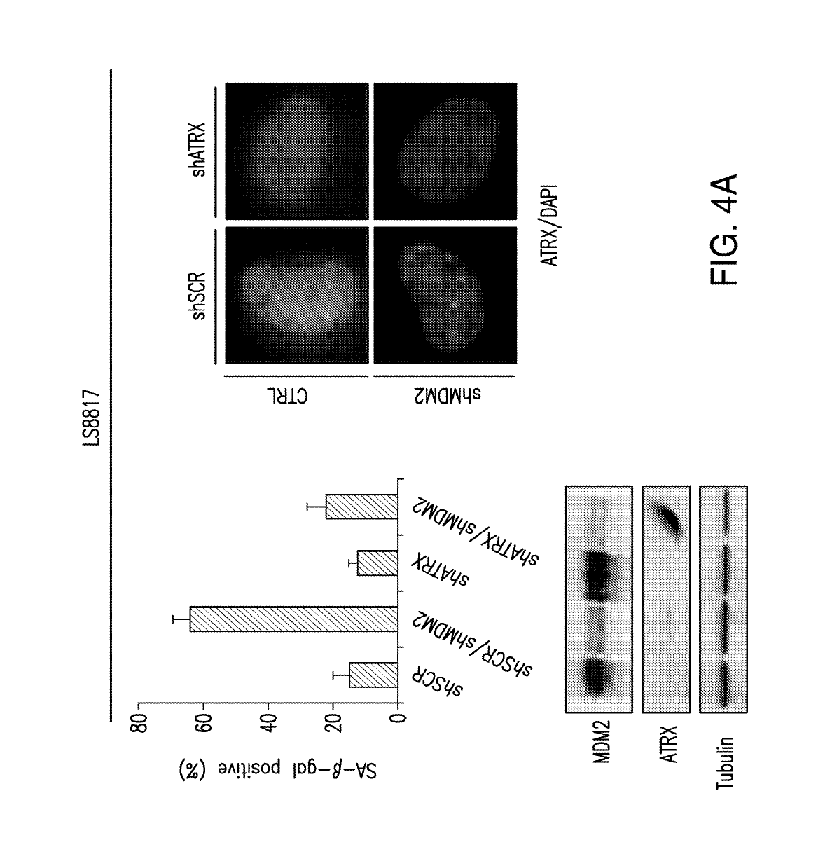

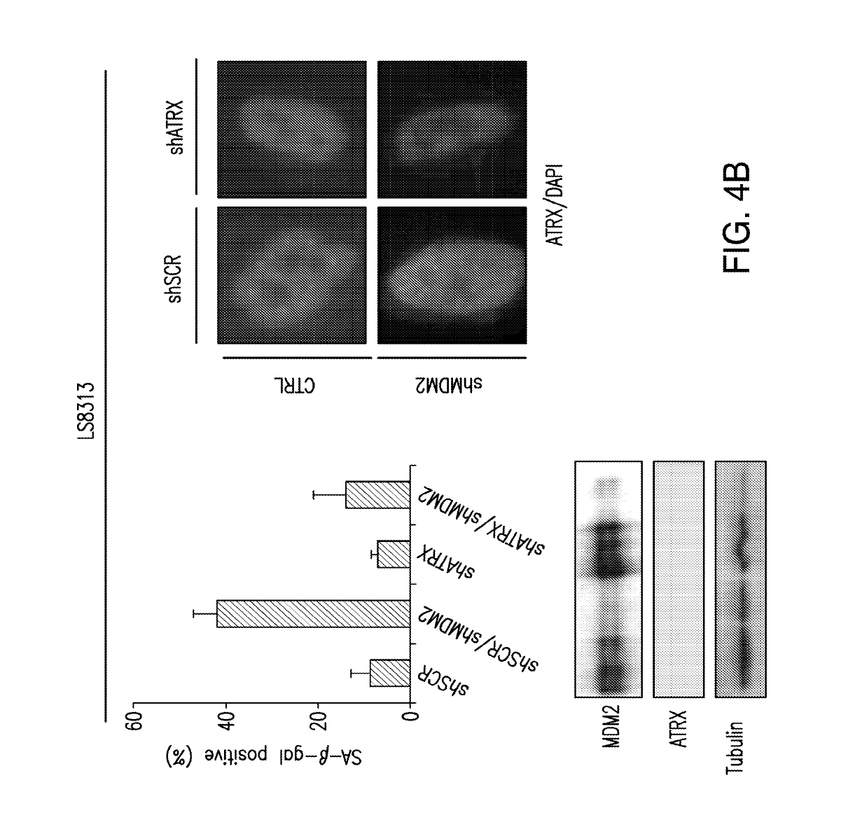

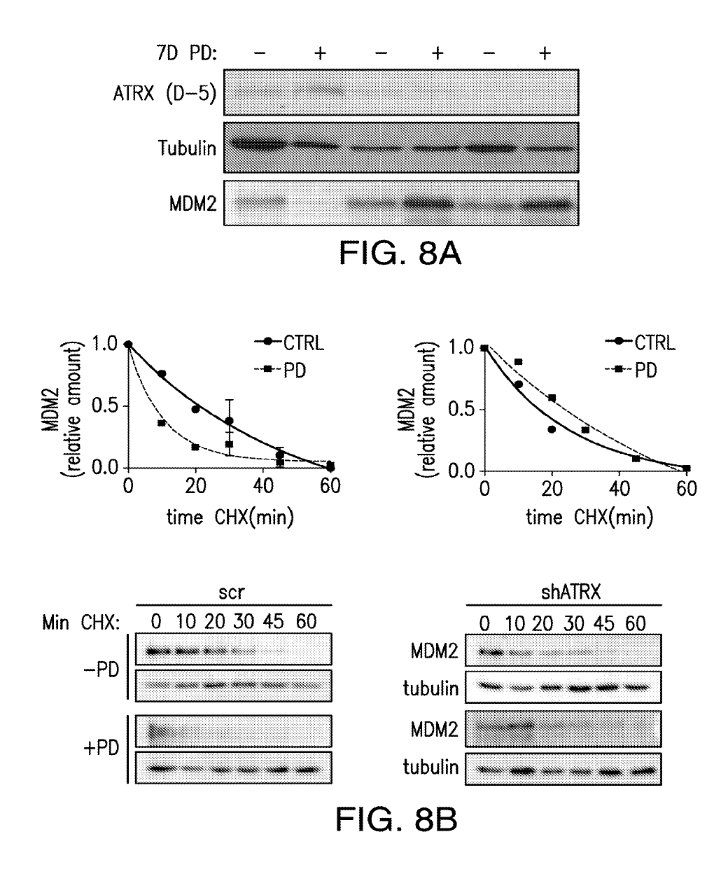

To determine if ATRX plays a role in the senescence pathway induced by treatment with a CDK4 inhibitor, the expression of ATRX was genetically manipulated in the responder cell line, LS8817. Knockdown of ATRX (FIG. 2) was performed in LS8817 cells that were treated with PD0332991. The presence of ATRX in scrambled control shRNA (shSCR)-treated cells and the ATRX shRNA-treated cells was detected by the Bethyl antibody. The accumulation of perinuclear associated β-galactosidase (SA-β-gal) shSCR-treated cells and the ATRX shRNA-treated cells was measured (FIG. 3). In LS8817 cells infected with shRNA directed to ATRX, the number of SA-β-gal positive cells observed after exposure to PD0332991 for seven days was greatly reduced as compared to PD0332991-treated LS8817 cells infected with shSCR, without serious consequences to cell proliferation as measured by BrdU (FIG. 3). Abrogating ATRX expression, as detected by the D5 antibody, prevented the loss of MDM2 that has been shown to accompany induction of senescence following treatment with PD0332991 (FIGS. 3 and 8A). As shown in FIG. 8B, reducing ATRX levels in the responder cell line L8817 prevented CDK4 inhibition-induced acceleration of MDM2 protein turnover (FIG. 8B). To determine if ATRX was also downstream of MDM2 loss, we subsequently infected the ATRX deficient cells with lentiviruses expressing shRNAs that targeted MDM2. In the responder cell line, LS8817, accumulation of SA-β-gal was still compromised even though the level of MDM2 was reduced (FIG. 4A). Similar results were seen in the LS8313 non-responder cells-ATRX knockdown prevented senescence induced by knocking down MDM2 (FIG. 4B). Thus, a form of ATRX was required for both PD0332991-induced loss of MDM2, and downstream of MDM2 loss to establish a senescent state.

### 6.3 Discussion

We have shown that ATRX was important for an INK4A- and p53-independent senescence program. First, a form of ATRX detectable by immunoblot using the Bethyl antibody was present in the responder cells and not in the non-responder cells. Reducing all forms of ATRX in responder and non-responder cells does not interfere with the ability of PD0332991 to induce cell cycle exit, but it does prevent the down-regulation of MDM2 in responders. Flow might ATRX contribute to MDM2 turnover? ATRX interacts with DAXX (Lewis et al., 2010). DAXX is required to bridge HAUSP, a deubiquitinase, to the MDM2 complex (Tang et al., 2006). In this MDM2/DAXX/HAUSP complex, MDM2 autoubiquitination is reduced and ubiquitination of other substrates is favored (Li et al., 2004; Meulmeester et al., 2005; Tang et al., 2006). Proteins that interact with DAXX can disrupt this complex. For example, upon DNA damage the activation of the DNA damage responsive kinases (ATM, ATR, or DNA-PK) enhance the ability of RASSF1A to interact with DAXX disrupting the MDM2/DAXX/HAUSP complex. This leads to MDM2 autoubiquitination and turnover (Song et al., 2008). ATRX may use an analogous mechanism in responder cells. Triggered by the inhibition of CDK4, ATRX could compete for DAXX driving the autoubiquitination and degradation of MDM2. Non-responder cells lack this form of ATRX and thus MDM2 would be stabilized. Given that the immunoblot detectable form is one type of ATRX in cells, we suspect that regulation may be more complex than simple titration and the relationship between ATRX and the MDM2/DAXX/HAUSP complex and its modulation by CDK4 inhibition warrants further investigation.

Secondly, reducing all forms of ATRX in both responder and non-responder cells prevents senescence following the loss of MDM2. This indicates that ATRX is required downstream for senescence. ATRX/DAXX facilitates the deposition of histone H3.3 into telomeric nucleosomes and pericentric heterochromatin (Clynes et al., 2013; Drane et al., 2010). There are other histone H3.3 chaperones, and at least one, the HIRA/ASF1 containing complex, is involved in the deposition of H3.3 in senescent cells (Banumathy et al., 2009; Zhang et al., 2007; Zhang et al., 2005). Recently, however, HIRA/ASF1 was shown to operate largely in cycling cells (Ray-Gallet et al., 2011) and many of the target genes are transcriptionally active (Pchelintsev et al., 2013), but the HIRA complex and histone H3.3 are involved in chromatin silencing in some situations as well (Sherwood et al., 1993; van der Heijden et al., 2007). Thus, these histone chaperone pathway could contribute to distinct local histone H3.3 enrichment in different contexts (Goldberg et al., 2010). ATRX/DAXX may play a significant role in the pathway induced by loss of MDM2.

### 7. EXAMPLE 2

### Modified Form of ATRX in Non-Responder Cells

This Example discloses the identification of the form of ATRX present in non-responder cells as compared to responder cells.

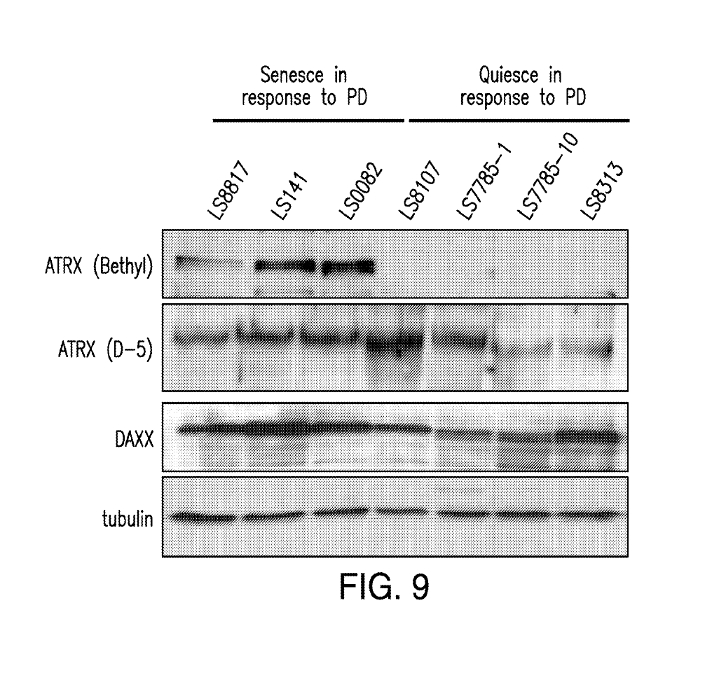

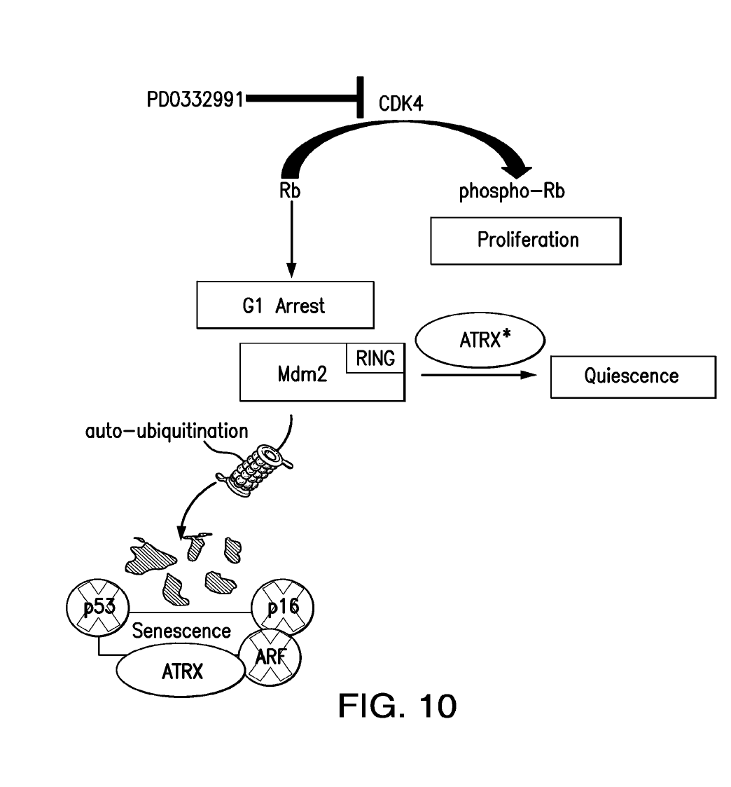

The poor correlation between ATRX and ALT prompted the measuring of ATRX with another antibody, the D5 antibody sold by Santa Cruz Biotechnology, Catalog No. sc-55584, that was raised to a different antigen than the Bethyl Antibody. ATRX using the D5 antibody was detected in all WD/DDLS cell lines, including the non-responder cells, e.g., LS8107 (FIG. 9). Without being bound to a specific theory, these results suggest that a modification of ATRX occluded the Bethyl antibody from detecting ATRX in non-responders (FIG. 10).

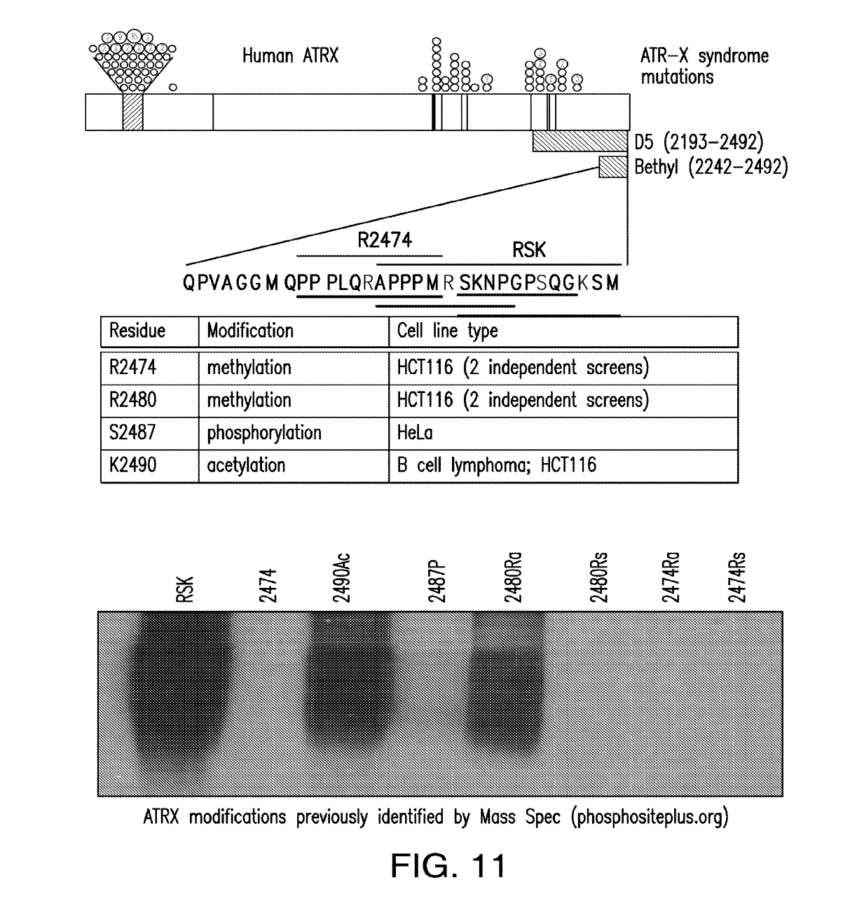

FIG. 11 shows the locations of the highly conserved N-terminal cysteine-rich domain and the C-terminal helicase-like domain of ATRX. The positions of missense mutations are indicated in circles and the number of times the mutation has been identified in unrelated individual is indicated in the relevant circles. As shown in FIG. 11, ATRX is a protein that has 2492 amino acids, and the ATRX-Dnmt3-Dnmt3L (ADD) domain of ATRX consists of amino acids 159-296 and the helicase domain consists of amino acids 1581-2205 of the ATRX protein. The D5 antibody was raised to a peptide having the amino acid sequence of amino acids 2193-2492 of ATRX; whereas, the Bethyl antibody was raised to a peptide having the amino acid sequence of amino acids 2193-2492 of ATRX.

To determine the form of ATRX that was not detectable by the Bethyl antibody, 8 peptides were generated, some of which contained post-translational modifications (FIG. 11 and Table 1). Table 1 shows the 8 peptides that were generated and tested for reactivity with the Bethyl antibody. All peptides were generated by Thermo Fisher Scientific/Pierce Biotechnology and coupled to the carrier KLH. All conjugates were provided at >98% purity as determined by HPLC. Peptide sequences were confirmed by mass spec. Peptides RSK, 2490Ac and 2480Ra reacted with the Bethyl antibody (FIG. 11). In contrast, peptides 2474, 2474Ra, 2474Rs, 2487P and 2480Rs did not react with the Bethyl antibody (FIG. 11), suggesting that the form of ATRX present in non-responders can be phosphorylated at 2487 and/or symmetrically methylated at 2480.

TABLE 1

| Peptide | Peptide Amino | Post-translational |
|---|---|---|
| Name | Acid Sequence | Modification of Peptide |
| RSK | APPMRSKNPGPSQGKSM | None |
| | (amino acids 2475-2492 | |
| | of ATRX) | |
| 2474 | PPPLQRAPPM | None |
| | (amino acids 2469-2479 | |
| | of ATRX) | |
| 2474Ra | PPPLQRAPPM | Asymmetrically methylated at arginine |
| | (amino acids 2469-2479 | (R), indicated in bold, which |
| | OF ATRX) | corresponds to amino acid 2474 of |
| | | ATRX |
| 2474Rs | PPPLQRAPPM | Symmetrically methylated at arginine |
| | (amino acids 2469-2479 | (R), indicated in bold, which |
| | of ATRX) | corresponds to amino acid 2474 of |
| | | ATRX |
| 2490Ac | SKNPGPSQGKSM | Acetylated at lysine (K), indicated in |
| | (amino acids 2481-2492 | bold, which corresponds to amino acid |
| | of ATRX) | 2490 of ATRX |
| 2487P | SKNPGPSQG | Phosphorylated at serine (S), indicated |
| | (amino acids 2481-2489 | in bold, which corresponds to amino |
| | of ATRX) | acid 2487 of ATRX |
| 2480Ra | APPPMRSKNP | Asymmetrically methylated at arginine |
| | (amino acids 2475-2484 | (R), indicated in bold, which |
| | of ATRX) | corresponds to amino acid 2480 of |
| | | ATRX |
| 2480Rs | APPPMRSKNP | Symmetrically methylated at arginine |
| | (amino acids 2475-2484 | (R), indicated in bold, which |
| | of ATRX) | corresponds to amino acid 2480 of |
| | | ATRX |

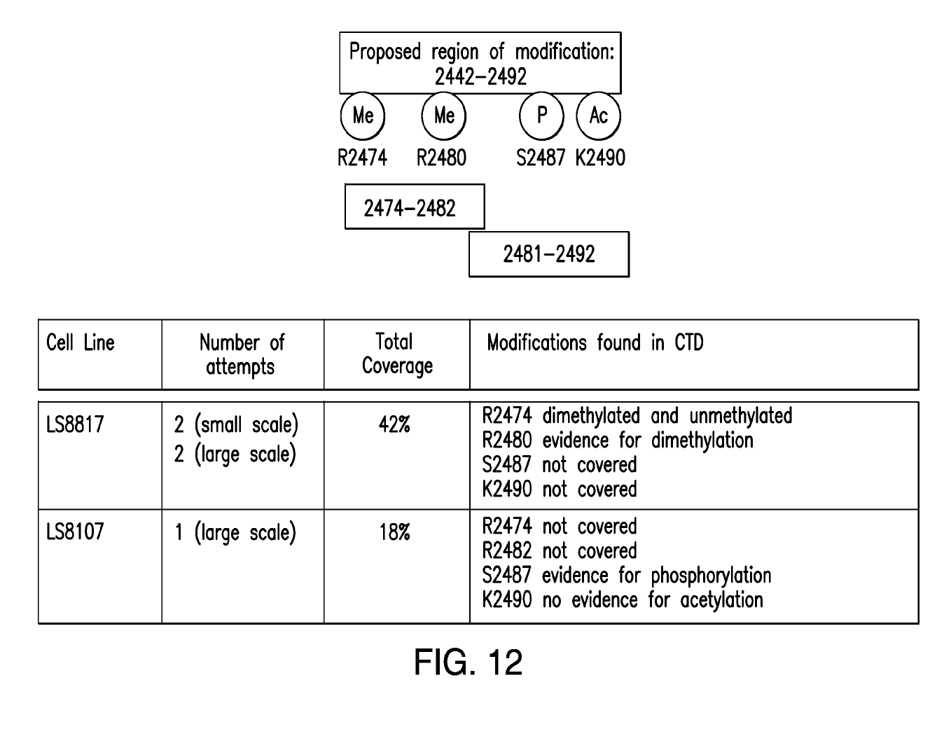

To further determine which modified form of ATRX is present in non-responders, mass spectrometry was performed on the non-responder cell line LS8107 and the responder cell line LS8817. In the responder cell line, unmethylation and dimethylation at amino acid R2474 of ATRX was detected (FIG. 12). Dimethylation at amino acid R2480 of ATRX was detected (FIG. 12). In non-responder cells, there was no evidence for acetylation at amino acid K2490 of ATRX (FIG. 12). However, phosphorylation of amino acid S2487 was detected in non-responder cells (FIG. 12). Without being bound to a specific theory, these results suggest that ATRX in the non-responder cells is phosphorylated at amino acid S2487.

### 8. EXAMPLE 3

### ATRX Foci in Response to CDK4 Inhibition

This Example discloses the change in the number of ATRX foci in response to a CDK4 inhibitor in responder cells as compared to non-responder cells.

As noted above, ATRX plays a role in senescence and has been shown to interact with PML, macroH2A, HP1 and historic H3.3 (see Eustermann et al., 2011; Lewis et al., 2010; Ratnakumar et al., 2012; Xue et al., 2003), which have been show to interact with HIRA/ASF. Immunofluorescence analysis using the Bethyl antibody was performed to determine if ATRX was recruited to foci. Immunofluorescence was performed as described in Example 1.

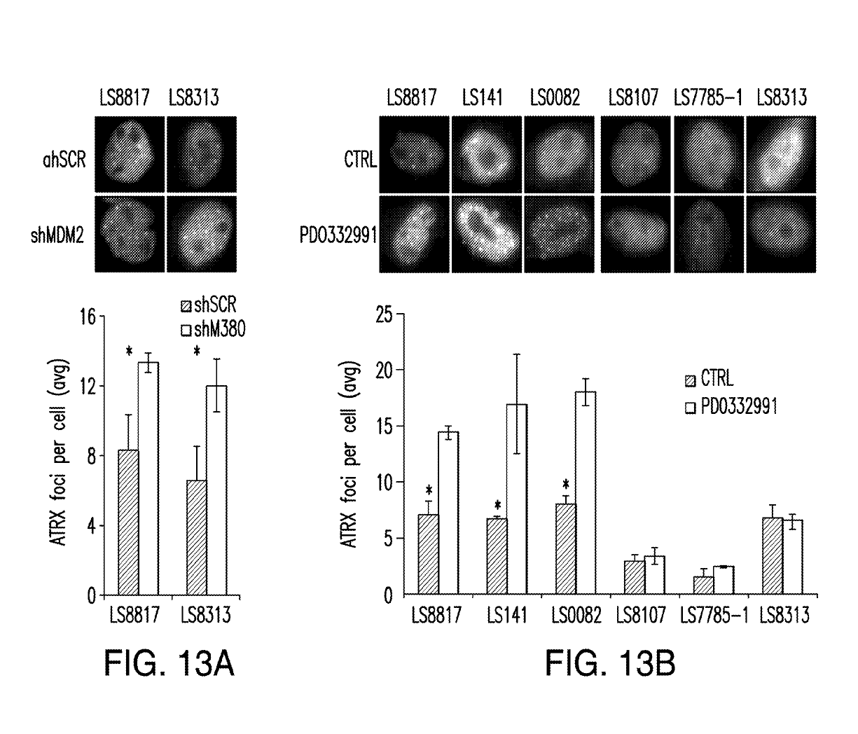

As shown in FIG. 13, the number of ATRX foci increased in senescent responder cells upon the loss of MDM2 (FIG. 13A) or in response to treatment with PD0332991 (FIG. 13B). There was no increase in the number of ATRX foci in PD0332991-treated non-responder cells, which did not senesce (FIG. 13B). These results suggest that ATRX foci can be used as a marker for senescence in response to CDK4 inhibition.

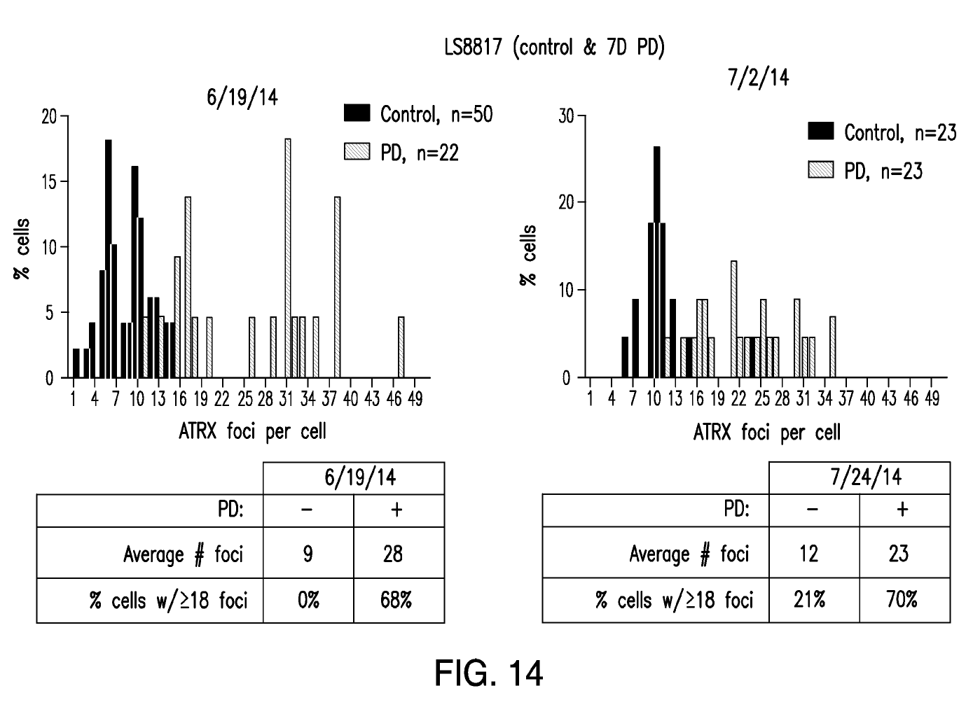

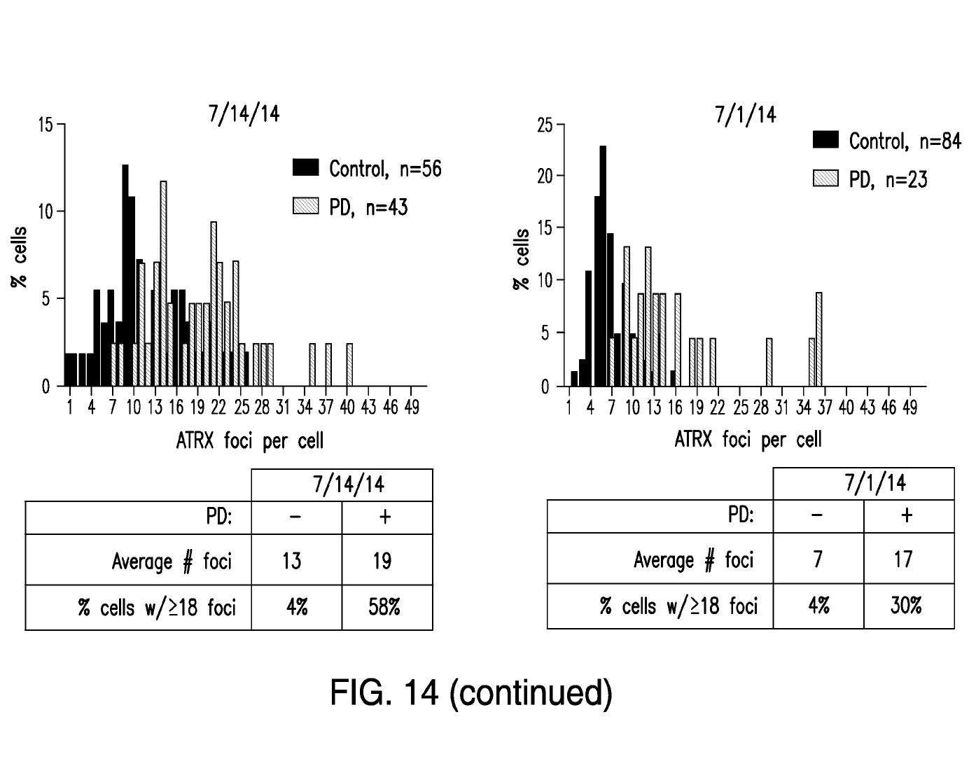

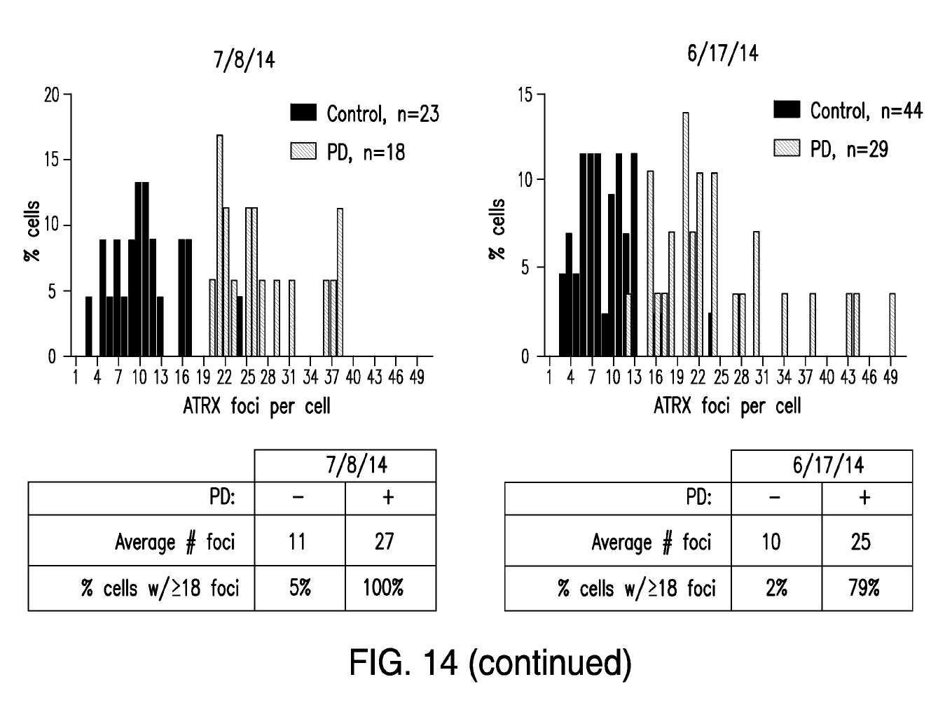

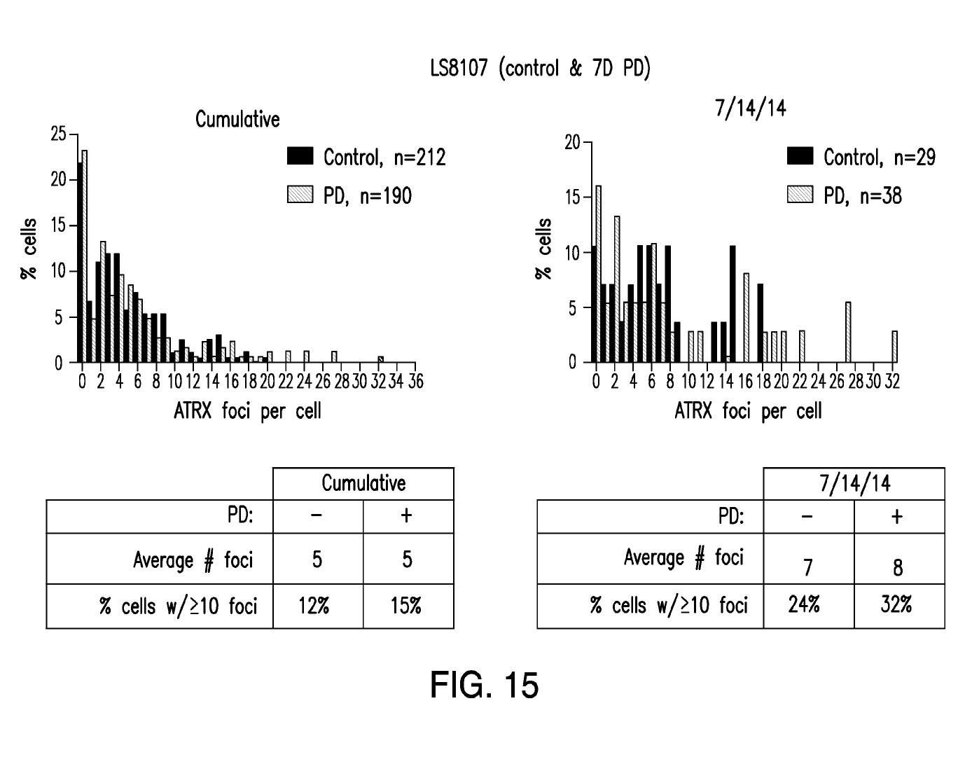

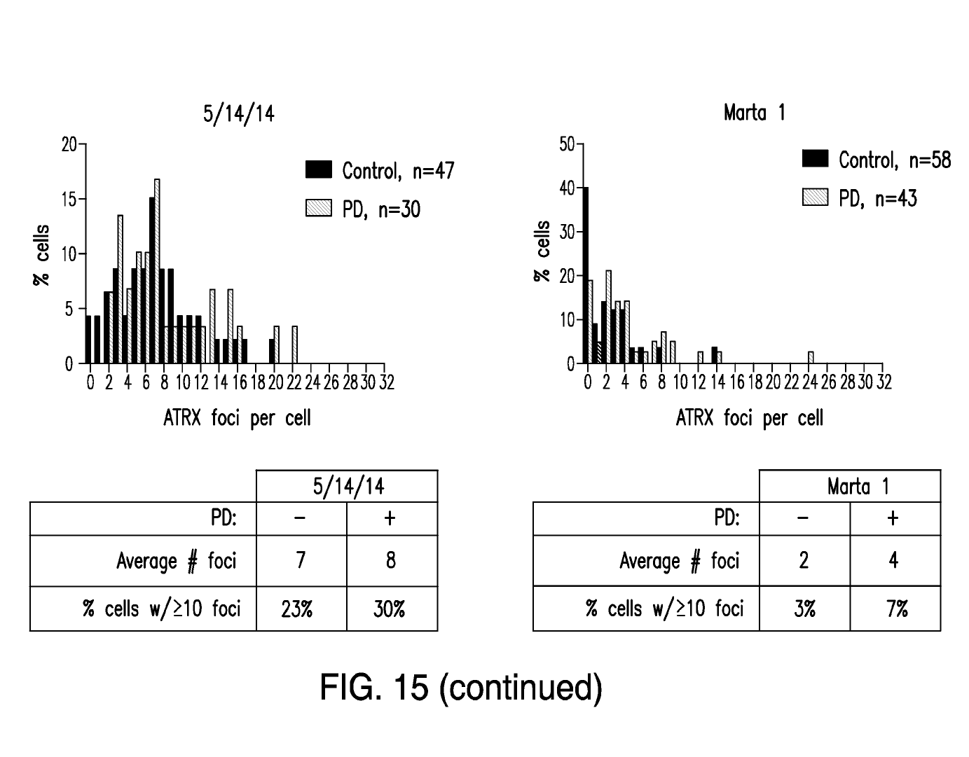

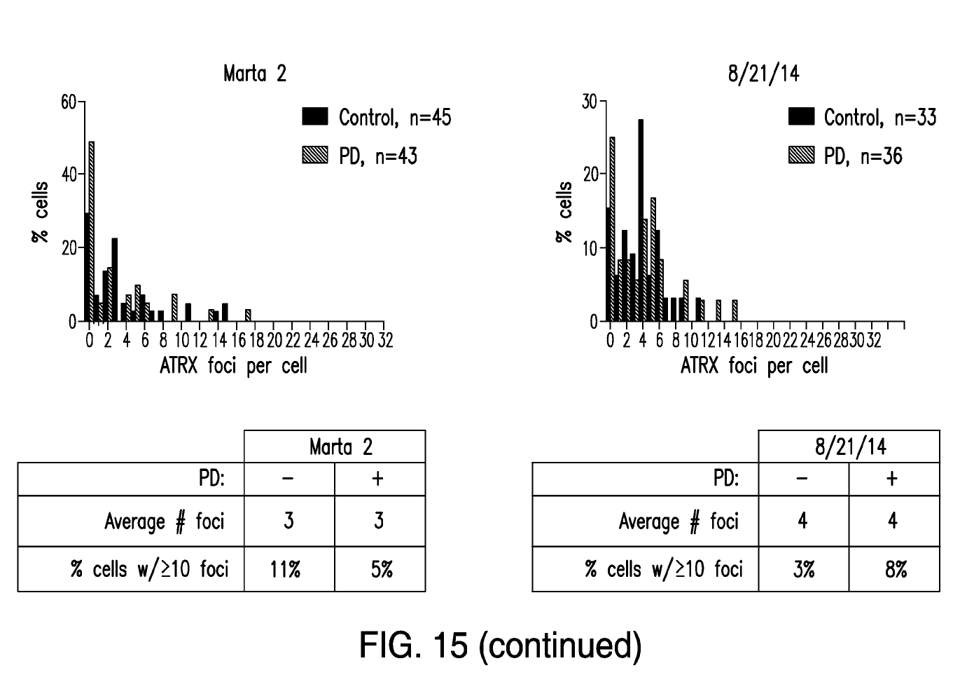

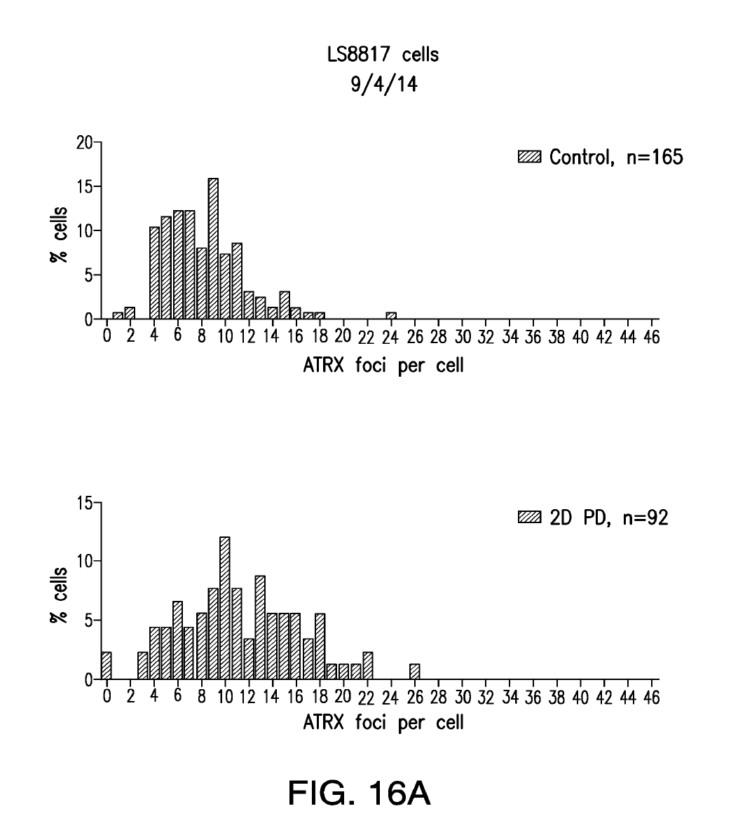

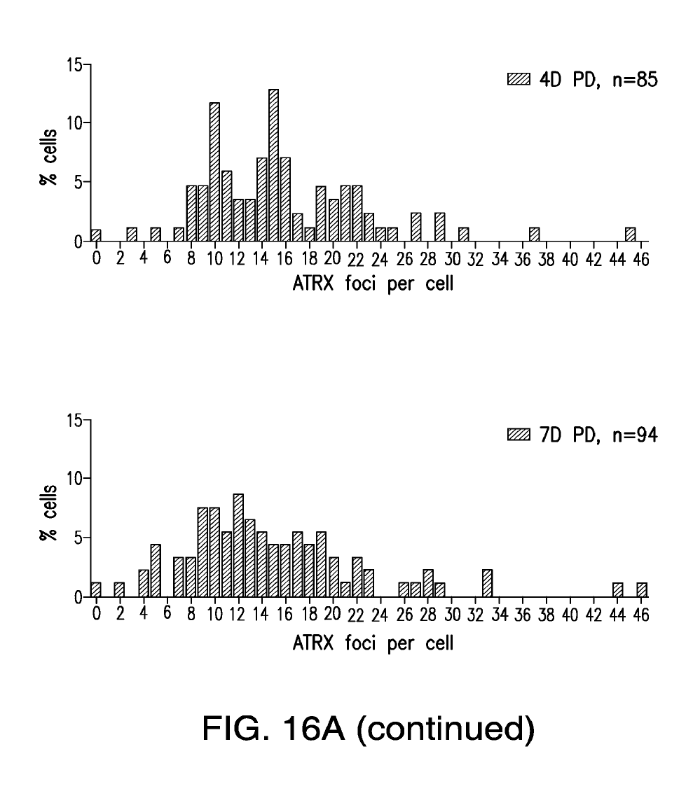

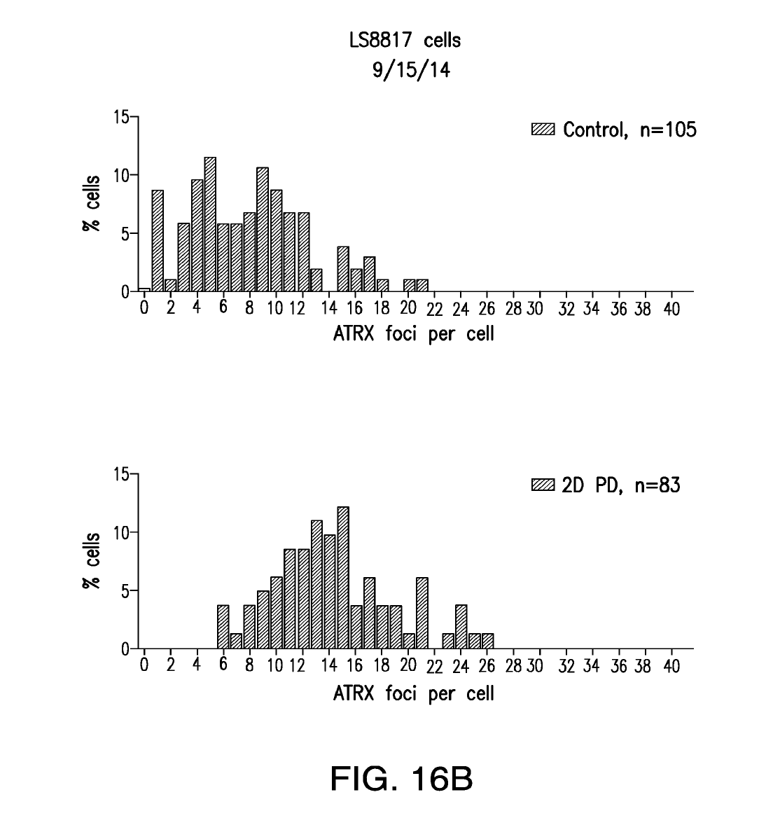

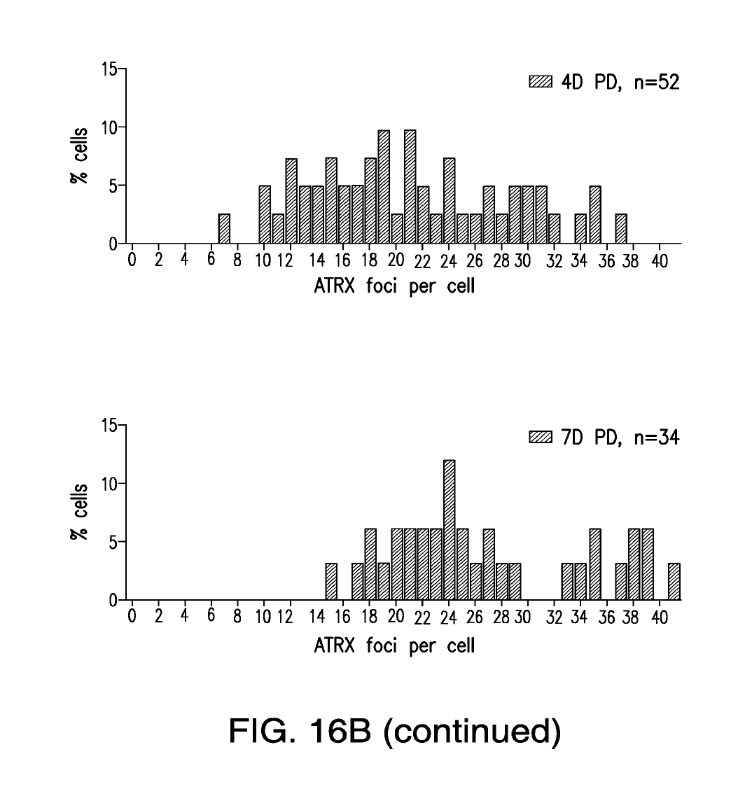

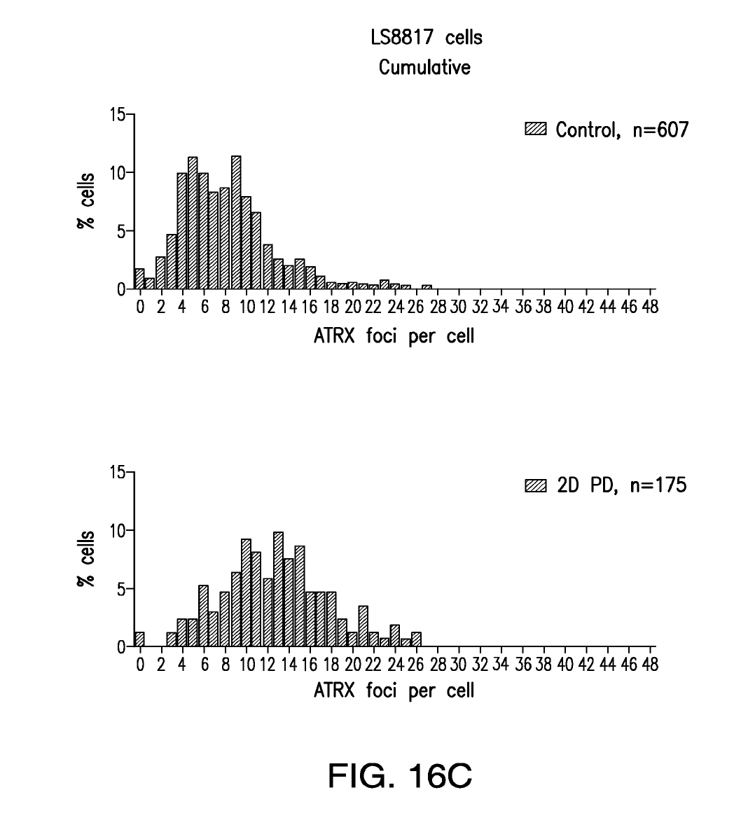

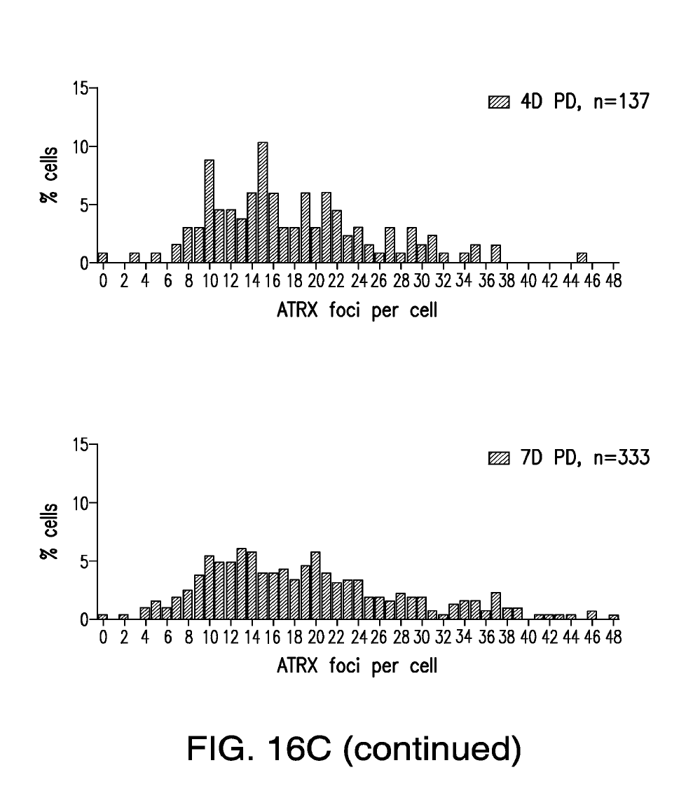

In the responder cell line LS8817, further analysis was performed to determine the average number of foci present in responder cells undergoing senescence in response treatment with PD0332991. As shown in FIG. 14, the number of ATRX foci in responder cells undergoing senescence was significantly more than in responder cells that did not undergo treatment with PD0332991. The distribution of the number of ATRX foci shifted to the right in responder cells treated with PD0332991 as compared to responder cells that were not treated. In addition, the number of cells that contained 18 or more ATRX foci greatly increased in response to PD0332991 treatment. In contrast, in the non-responder cell line LS8107, the average number of ATRX foci per cell did not change upon treatment with PD0332991 when compared with the non-responders that were not treated with PD0332991 (FIG. 15). As shown in FIG. 16A-C, the increase in the number of ATRX foci in response to PD0332991 treatment in responder cells can be observed as early as day 2 subsequent to PD0332991 treatment.

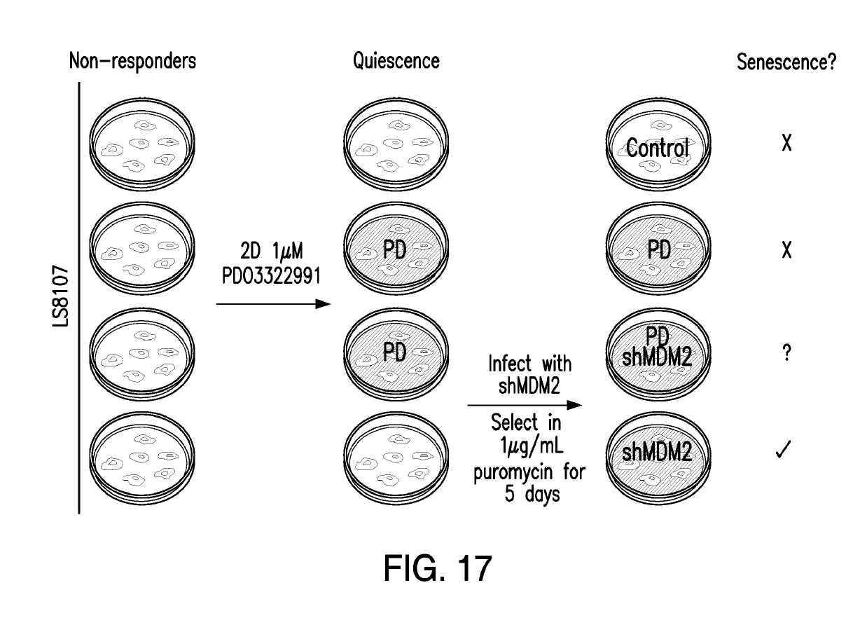

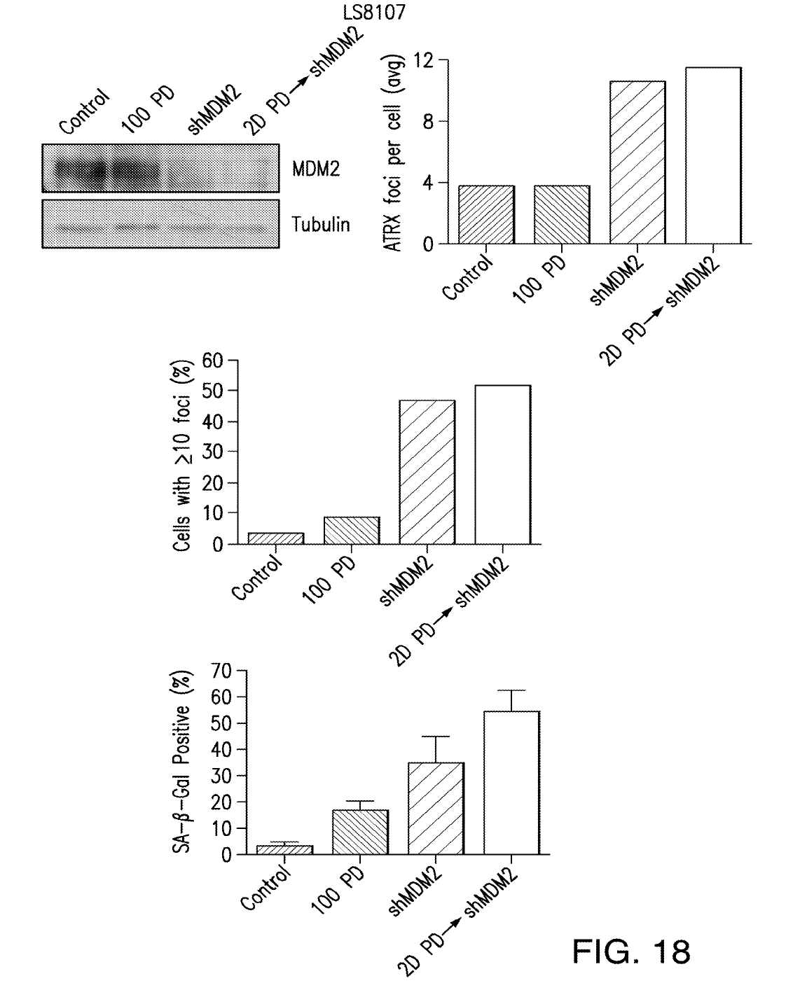

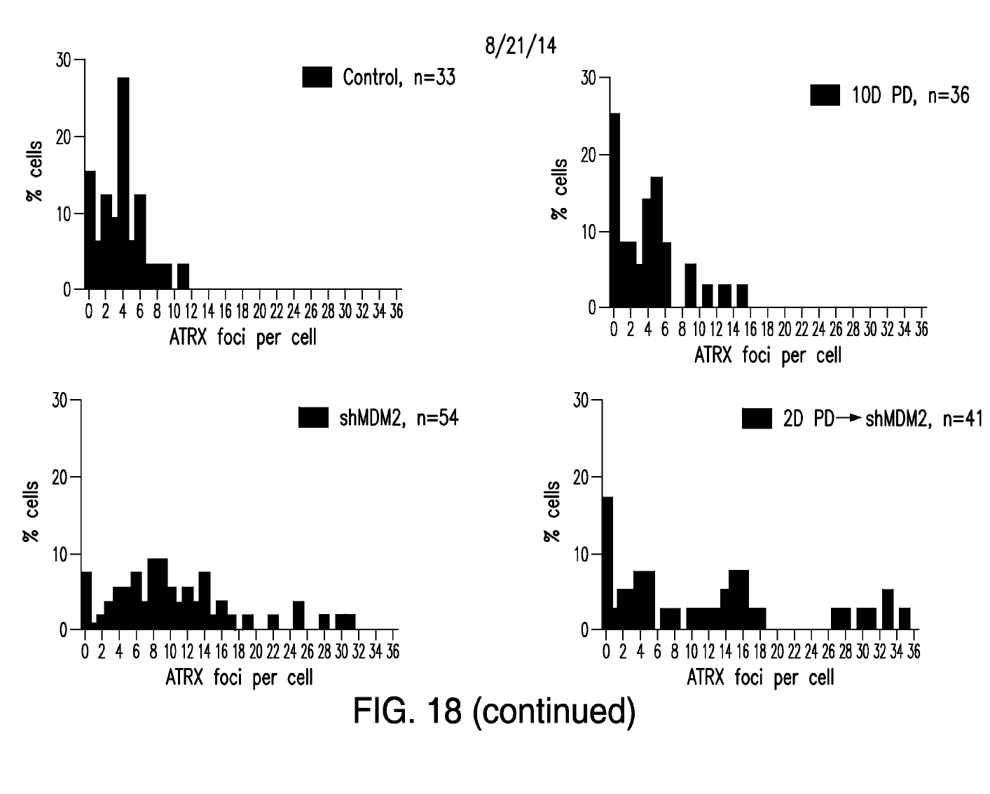

To determine whether ATRX foci can be used as a marker for indicating if non-responders underwent senescence in response to treatment with second drug in combination with a CDK inhibitor, non-responder cells, e.g., LS8107, was treated with PD0332991 followed by treatment with shRNA directed to MDM2 2 days post-PD0332991 treatment (FIG. 17). As shown in FIG. 18, the treatment of cells with PD0332991 followed by shMDM2 resulted in a significant increase in the number of ATRX foci within non-responders cells compared to cells that were not treated with PD0332991 or were treated with P130332991 alone. These results indicate that ATRX can be used a marker for indicating whether non-responder cells undergo senescence in response to CDK4 inhibition in conjunction with a second drug, e.g., MDM2 shRNA.

### 9. EXAMPLE 4

### Generation of Anti-ATRX Antibodies

This Example discusses the methods by which the disclosed antibodies were generated.

Mice were immunized with the peptides disclosed in Example 2 via intraperitoneal injections. The mice were immunized at least four times over a period of two months. Mice were initially injected and boosted with the 2487P peptide disclosed in Example 2 (which has the amino acid sequence SKNPGPSQG, where S is phosphorylated; SEQ ID NO:4). These mice were subsequently boosted twice with a peptide having the amino acid sequence SKNPGPSQGKSM, where S is phosphorylated (SEQ ID NO:5) to generate antibodies that bind ATRX that is phosphorylated at S2487.

Additional mice were injected with the 2480Rs peptide disclosed in Example 2 (which has the amino acid sequence APPPMRSKNP, or fragment thereof, where R is symmetrically dimethylated; SEQ ID NO:6). These mice were subsequently boosted three times with the same peptide to generate antibodies that hind ATRX that is symmetrically dimethylated at R2480.

### 10. REFERENCES

Banumathy et al., Human UBN1 is an ortholog of yeast Hpc2p and has an essential role in the HIRA/ASF1a chromatin-remodeling pathway in senescent cells, Mal. Cell Biol. 29(3):758-70 (2009).
Clynes et al., The chromatin remodeller ATRX: a repeat offender in human disease, 38(9):461-6 (2013).
Drane et al., The death-associated protein DAXX is a novel histone chaperone involved in the replication-independent deposition of H3.3, Genes and Dev. 24(12):1253-65 (2010).
Eusteunann et al., Combinatorial readout of histone H3 modifications specifies localization of ATRX to heterochromatin, Nat. Struct. Mol. Biol. 18(7):777-82 (2011).
Goldberg et al., Distinct factors control histone variant H3.3 localization at specific genomic regions. 140(5):678-91 (2010).
Heaphy et al., Altered telomeres in tumors with ATRX and DAXX mutations, Science 333(6041):425 (2011).
Henson et al., A robust assay for alternative lengthening of telomeres in tumors shows the significance of alternative lengthening of telomeres in sarcomas and astrocytomas, Clinical Cancer Res. 11(1):217-25 (2005).
Johnson and Broccoli, Telomere maintenance in sarcomas, Curr. Opin. Oncol. 19(4):377-82 (2007).
Lewis et al., Daxx is an H3.3-specific histone chaperone and cooperates with ATRX in replication-independent chromatin assembly at telomeres, Proc. Natl. Acad. Sci. 107(32):14075-80 (2010).
Li et al., A dynamic role of HAUSP in the p53-Mdm2 pathway, Mol Cell. 13(6):879-86 (2004).
Lovejoy et al. Loss of ATRX, genome instability, and an altered DNA damage response are hallmarks of the alternative lengthening of telomeres pathway, PLoS Genetics 8(7):e1002773 (2012).
Luciani et al., PMT, nuclear bodies are highly organised DNA-protein structures with a function in heterochromatin remodelling at the G2 phase, J, Cell Science 19(Pt 12):2518-31 (2006).
McDowell et al., Localization of a putative transcriptional regulator (ATRX) at pericentromeric heterochromatin and the short arms of acrocentric chromosomes, Proc. Natl. Acad. Sci. 96(24):13983-8 (1999).
Meulmeester et al., ATM-mediated phosphorylations inhibit Mdmx/Mdm2 stabilization by HAUSP in favor of p53 activation, Cell Cycle 4(9):1166-70 (2005).
Pchelintsev et al., Placing the HIRA histone chaperone complex in the chromatin landscape. Cell Resp. 3(4):1012-9 (2013).
Pereira and Ferriera, Sowing the seeds of cancer: telomeres and age-associated tumorigenesis, Curr. Opin. Oncol. 25(1):93-8 (2013).
Picketts et al., ATRX encodes a novel member of the SNF2 family of proteins: mutations point to a common mechanism underlying the ATR-X syndrome, Am. J. Human Genet. 5(12):1899-907 (1996).
Ratnakumar et al., ATRX-mediated chromatin association of histone variant macroH2A1 regulates α-globin expression, Genes and Dev. 26:433-438 (2012).
Ray-Gallet et al., Dynamics of histone H3 deposition in vivo reveal a nucleosome gap-filling mechanism for H3.3 to maintain chromatin integrity, Mol. Cell 44(6):928-41 (2011).
Sherwood et al., Characterization of HIR1 and HIR2, two genes required for regulation of histone gene transcription in Saccharomyces cerevisiae, Mol. Cell Biol. 13(1):28-38 (1993).
Slatter et al., Pilocytic astrocytomas have telomere-associated promyelocytic leukemia bodies without alternatively lengthened telomeres, Am. J. of Path. 177(6):2694-700 (2010).
Song et al., The tumour suppressor RASSF1A promotes MDM2 self-ubiquitination by disrupting the MDM2-DAXX-HAUSP complex, EMBO J. 27(13):1863-74 (2008).
Tang et al., Critical role for Daxx in regulating Mdm2, Nat. Cell Biol. 8(8):855-62 (2006).
United States Patent Application No. 2004/0141977 by Paz.
van der Heijden et al., Chromosome-wide nucleosome replacement and H3.3 incorporation during mammalian meiotic sex chromosome inactivation, Nat Genet. 39(2):251-8 (2007).
Xue et al., The ATRX syndrome protein forms a chromatin-remodeling complex with Daxx and localizes in promyelocytic leukemia nuclear bodies, Proc. Natl. Acad. Sci. 100(19):10635-40 (2003).
Zhang et al., Formation of MacroH2A-containing senescence-associated heterochromatin foci and senescence driven by ASF1a and HIRA, Dev. Cell. 8(1):19-30 (2005).
Zhang et al., Molecular dissection of formation of senescence-associated heterochromatin foci, Mol Cell Biol. 27(6):2343-58 (2007).

Various references are cited herein, the contents of which are hereby incorporated by reference in their entireties.

1. (canceled)

2. (canceled)

3. (canceled)

4. (canceled)

5. (canceled)

6. (canceled)

7. (canceled)

8. (canceled)

9. (canceled)

10. (canceled)

11. A method for treating a subject having a cancer, comprising, obtaining a sample of the cancer and determining, in the sample, the expression level of an ATRX biomarker using an ATRX-specific antibody, where if expression of the ATRX biomarker is detected, then treating the subject with a therapeutically effective amount of a CDK4 inhibitor,
wherein the ATRX-specific antibody is an antibody sold by Bethyl, Catalog No. A301-045A (“the '045 Ab”), a fragment thereof or an antibody that competitively inhibits binding of the '045 Ab to ATRX.

12. The method of claim 11, wherein the level of an ATRX biomarker is determined prior to treating the subject with CDK4 inhibitor.

13. The method of claim 11, wherein the cancer is liposarcoma, glioma, glioblastoma, or breast cancer.

14. The method of claim 11, wherein the expression of the ATRX biomarker is determined by Western Blot.

15. (canceled)

16. (canceled)

17. (canceled)

18. (canceled)

19. (canceled)

20. (canceled)

21. A method for producing an anti-cancer effect, in a subject, by a CDK4 inhibitor, comprising, obtaining a sample of the subject's cancer prior to treatment with a CDK4 inhibitor, and determining, in one or more cancer cell from the sample, the expression level of an ATRX biomarker using an ATRX-specific antibody, where if expression of the ATRX biomarker is detected in the one or more cancer cell, then initiating treatment of the subject with a therapeutically effective amount of a therapeutic agent which is not a CDK4 inhibitor,
wherein the ATRX-specific antibody is selected from the group consisting of Ab3, Ab4 or combinations thereof.

22. The method of claim 21, wherein the cancer is liposarcoma, glioma, glioblastoma, or breast cancer.

23. The method of claim 21, wherein the expression of the ATRX biomarker is determined by Western Blot using the ATRX-specific antibody.

24. (canceled)

25. (canceled)

26. (canceled)

27. (canceled)

28. (canceled)

29. (canceled)

30. (canceled)

31. (canceled)

32. (canceled)

33. (canceled)

34. A method for producing an anti-cancer effect, in a subject, by a CDK4 inhibitor, comprising, obtaining a sample of the subject's cancer prior to treatment with a CDK4 inhibitor, and determining, in one or more cancer cell from the sample, the expression level of an ATRX biomarker, where if expression of the ATRX biomarker is detected in the one or more cancer cell, then initiating treatment of the subject with a therapeutically effective amount of a therapeutic agent which is not a CDK4 inhibitor,
wherein the ATRX biomarker is an ATRX protein that is phosphorylated at S2487.

35. The method of claim 34, wherein the cancer is liposarcoma, glioma, glioblastoma, or breast cancer.

36. The method of claim 34, wherein the expression of the ATRX biomarker is determined by Western Blot using an ATRX-specific antibody.

37. (canceled)

38. (canceled)

39. (canceled)

40. (canceled)

41. (canceled)

42. (canceled)

43. (canceled)

44. A kit for determining whether an anti-cancer effect is likely to be produced in a cancer by a CDK4 inhibitor, comprising a means for detecting an ATRX biomarker.

45. The kit of claim 44, wherein the means for detecting an ATRX biomarker comprises one or more packaged probe, arrays/microarrays, ATRX-specific antibodies and/or beads.

46. The kit of claim 45, wherein the one or more ATRX-specific antibodies comprises an antibody sold by Bethyl, Catalog No. A301-045A (“the '045 Ab”), a fragment thereof, or an antibody that competitively inhibits binding of the '045 Ab to ATRX.

47. The kit of claim 45, wherein the one or more ATRX-specific antibodies comprises an isolated antibody, or an antigen-binding portion thereof, which specifically binds to ATRX, wherein the antibody or antigen-binding portion thereof binds to an ATRX epitope within a fragment of ATRX comprising the amino acid sequence SKNPGPSQG, or fragment thereof, where S is phosphorylated (SEQ ID NO: 4).

48. The kit of claim 45, wherein the one or more ATRX-specific antibodies comprises an isolated antibody, or an antigen-binding portion thereof, which specifically binds to ATRX, wherein the antibody or antigen-binding portion thereof binds to an ATRX epitope within a fragment of ATRX comprising the amino acid sequence SKNPGPSQGKSM, or fragment thereof, where S is phosphorylated (SEQ ID NO: 5).

49. An isolated antibody, or an antigen-binding portion thereof, which specifically binds to ATRX, wherein the antibody or antigen-binding portion thereof binds to an ATRX epitope within a fragment of ATRX comprising the amino acid sequence SKNPGPSQG, or fragment thereof, where S is phosphorylated (SEQ ID NO: 4).

50. An isolated antibody, or an antigen-binding portion thereof, which specifically binds to ATRX, wherein the antibody or antigen-binding portion thereof binds to an ATRX epitope within a fragment of ATRX comprising the amino acid sequence SKNPGPSQGKSM, or fragment thereof, where S is phosphorylated (SEQ ID NO: 5).

51. The isolated antibody, or an antigen-binding portion thereof, of claim 49, wherein the antibody, or an antigen-binding portion thereof, is not the D5 antibody sold by Santa Cruz Biotechnology, Catalog No. sc-55584.

52. An assay for identifying compounds for use in combination with a CDK4 inhibitor, comprising:
(a) treating one or more cells that are not responsive to CDK4 inhibition with a CDK4 inhibitor;
(b) treating the one or more CDK4-treated cells with a putative compound; and
(c) determining the number of ATRX foci per cell in the one or more CDK4 and putative compound-treated cells, as compared to a reference, where an increase in the number of ATRX foci per cell compared to a reference is indicative that the putative compound is likely to enhance the effectiveness of the CDK4 inhibitor for treatment of a subject with cancer.

53. The method of claim 11, wherein the cancer is liposarcoma.
